(19) United States
(12) Patent Application Publication
(10) Pub. No.: US 2022/0119436 A1
(43) Pub. Date: Apr. 21, 2022

(54) METHOD FOR IMPROVED PRODUCTION OF ISOMALT

(71) Applicant: SÜDZUCKER AG, Mannheim (DE)

(72) Inventors: ALIREZA HAJI BEGLI, Ramsen (DE); CHRISTINE KRÖNER, Kindenheim (DE); RALPH CARTARIUS, Offenau (DE)

(21) Appl. No.: 17/275,146

(22) PCT Filed: Sep. 10, 2019
(86) PCT No.: PCT/EP2019/074102

§ 371 Date: Mar. 10, 2021

(30) Foreign Application Priority Data
Sep. 11, 2018 (EP) 18193756.6

(51) Int. Cl. C07H 1/00; A23L 27/30; C07H 15/04

(52) U.S. Cl. CPC C07H 1/00; A23V 2002/00; C07H 15/04; A23L 27/34

(57) ABSTRACT

The present invention relates to an improved method for the production of isomalt from an isomaltulose- and sucrose-containing carbohydrate mixture.

The present invention relates to a method for the improved production of isomalt, in particular a particularly storage-stable and pure isomalt, from an isomaltulose- or isomaltulose- and trehalulose- and sucrose-containing carbohydrate mixture, which is characterized in particular by improved efficiency, process stability and reaction selectivity and yield, and the isomalt produced in this way.

Isomalt (hydrogenated isomaltulose) is a sugar substitute, the main components of which comprise 1,6-GPS (6-O-α-D-glucopyranosyl-D-sorbitol) and 1,1-GPM (1-O-α-D-glucopyranosyl-D-mannitol). Due to its non-cariogenic, tooth-friendly properties, its low calorific value and its low glycemic effect, isomalt has numerous nutritional-physiological advantages. According to the Joint FAO/WHO Expert Committee on Food Additives (JECFA) specification (69th JECFA (2008), published in FAO JECFA Monographs 5 (2008)), isomalt comprises at least 98% by weight hydrogenated mono- and disaccharides (namely 1,6-GPS, 1,1-GPM, 1,1-GPS (1-O-α-D-glucopyranosyl-D-sorbitol), sorbitol and mannitol) and a maximum of 2% by weight secondary components, wherein this isomalt contains at least 86% by weight 1,6-GPS and 1,1-GPM and 0 to 0.3% by weight reducing sugars (each based on the dry matter of the isomalt). An isomalt according to the JECFA specification that is considered to be “sugar-free” under food law, which also meets the European purity criteria for additives and requirements from standard works, such as the FCC (Food Chemical Codex) as well as relevant isomalt monographs in drug books in Europe, the USA or Japan, is moreover distinguished by the fact that some of the secondary components that may be present are each present in a maximum amount of 0.5% by weight and that a total of 0.5% by weight of reducing and non-reducing sugars are present (for example mono- and disaccharides) (each based on the dry matter of the isomalt).

Isomalt is known in different variants. As can be seen from DE 195 32 396 C2, for example, isomalt can be present as an almost equimolar mixture of 1,6-GPS and 1,1-GPM. Other isomalt variants are characterized, for example, by an increased 1,6-GPS content (so-called 1,6-GPS-enriched isomalt), in particular by the fact that a 1,6-GPS content of 57 to 99% by weight and a 1,1-GPM proportion of 43 to 1% by weight (based on the main isomalt components) is present and, due to this composition, are endowed with increased sweetening power and solubility. As known from DE 195 23 008 A1 or EP 2 361 255 B1, such 1,6-GPS-enriched mixtures can be produced using ruthenium catalysts.

Isomalt is usually produced in a two-step process, in which isomaltulose (also known as palatinose) is first obtained from sucrose by enzyme-catalytic means using sucrose-glucosylmutases and then the isomaltulose is hydrogenated by metal-catalytic means with hydrogen (H₂).

In the enzymatic isomerization of sucrose to isomaltulose, the 1,2-glycosidic linkage of glucose and fructose in sucrose becomes a 1,6-glycosidic linkage in isomaltulose. As a result, the non-reducing sucrose, which does not contain any free aldehyde or keto group, becomes reducing isomaltulose, a keto-disaccharide with a free keto group. The 1,6-glycosidic linkage between glucose and fructose in isomaltulose is, compared to the 1,2-glycosidic linkage in sucrose, considerably more hydrolysis-stable, which means that the isomaltulose is considerably more resistant to acids and microbial fermentation.

On an industrial scale, isomalt is produced, as can be seen from EP 0 625 578 A1, starting from sucrose or a sucrose-containing starting mixture by subjecting the sucrose or the sucrose-containing starting mixture to an enzymatic isomerization reaction using a sucrose-glucosyl mutase, which leads to the formation of a so-called “isomerized sucrose”, which, as a rule, contains not only isomaltulose but also unreacted residual amounts of sucrose, also known as “residual sucrose”. This “isomerized sucrose” obtained from the isomerization of the sucrose or sucrose-containing starting mixture, hereinafter also referred to as isomaltulose- and sucrose-containing carbohydrate mixture, is then subjected to a hydrogenation reaction to reduce the isomaltulose and trehalulose possibly also present in the carbohydrate mixture with another isomerization of the sucrose to the disaccharide alcohols 1,6-GPS and 1,1-GPM and optionally 1,1-GPS (1-O-α-D-glucopyranosyl-D-sorbitol, from trehalulose).

If the hydrogenation is carried out under normal process conditions in a neutral or alkaline medium, usually using Raney nickel catalysts (EP 0 625 578 A), the residual sucrose amount present in the “isomerized sucrose” remains unchanged because the sucrose, due to its chemical structure, is non-reducing and can neither be hydrolytically split nor hydrogenated. A direct production of an isomalt with a 1,6-GPS content which is higher than the 1,1-GPM content is, however, not easily possible by means of such a procedure. However, if sucrose is hydrogenated under normal process conditions in an acidic medium, the acid-labile sucrose—as can be seen from U.S. Pat. Nos. 4,072,628, 3,963,788, or 4,950,812, in which the hydrogenation of sucrose using ruthenium-based catalysts is disclosed—is hydrolyzed to glucose and fructose, which as reducing sugars are hydrogenated to sorbitol and mannitol. In this case one speaks of a “splitting hydrogenation” of sucrose. The isomaltulose, on the other hand, remains stable even under acidic hydrogenation conditions due to its high acid stability and, as can be seen from DE 195 23 008 A1, can easily be hydrogenated directly to isomalt, in particular using a ruthenium-based catalyst.

From EP 2 361 255 B1 it is known to hydrogenate a mixture of isomaltulose and “residual sucrose” by means of a ruthenium-based catalyst for the production of isomalt, wherein the sucrose is split hydrogenated to sorbitol and mannitol via glucose and fructose under conditions that enable sucrose hydrolysis and the isomaltulose is hydrogenated to 1,1-GPM and 1,6-GPS. The use of a ruthenium-based catalyst leads in particular to the production of an isomalt with a 1,6-GPS content which is higher than the 1,1-GPM content, which is advantageous for certain applications (DE 195 23 008 A1). Because of the method conditions used in the ruthenium-based methods described, an isomalt produced in this way has, however, a large number and amount of by-products that is undesirable for many applications. Under the specific method conditions of Examples 1 to 4 shown in the publication, in particular temperatures of 90 and 120° C. and a pressure of 60 bar, the isomaltulose is only converted at a comparatively low conversion rate or energetically unfavorable high temperatures are used. In addition, the lifetime of the catalyst used is shortened, which leads to increased production costs. In addition, the presence of these by-products also reduces the purity of the product obtained and thus its marketability. On the one hand, the splitting of sucrose into glucose and fructose and the associated hydrogenation to form the by-products sorbitol and mannitol is desirable in that, due to nutritional requirements, undesired larger amounts of residual sucrose in the isomalt to be provided are avoided. On the other hand, the monosaccharide alcohols sorbitol and mannitol obtained from the splitting hydrogenation, in particular sorbitol, are often technologically disadvantageous since, due to their hygroscopicity, they make drying steps that may become necessary in the isomalt production method difficult, or at least make them more expensive and their presence in some applications, for example in hard candies, due to their hygroscopicity leads to undesired product properties such as stickiness, poor dimensional stability, such as deliquescence of the products (EP 1 776 015 B1), and poor shelf life.

In particular in cases in which—usually obtained enzymatically from sucrose or sucrose-containing starting mixtures on an industrial scale—sucrose-containing isomaltulose mixtures with a very low residual sucrose content are to be hydrogenated to isomalt, it can therefore be desirable that the sucrose passes through the hydrogenation method unchanged so that no sorbitol and mannitol are formed from it.

The increased hygroscopicity of the hydrogenated splitting products of sucrose, in particular sorbitol, in relation to sucrose itself as well as to isomalt, also reduces the shelf life of isomalt, in particular in humid and hot climatic regions, and makes it difficult to use isomalt in said humid and hot climatic regions, in particular in sweets, such as hard candies, and in particular in medicines that require complex packaging.

Although a number of processes for the production of isomalt from isomaltulose and sucrose-containing carbohydrate mixtures are known, which were obtained by enzymatic conversion of sucrose or sucrose-containing starting mixtures, there is still a need for methods that use isomalt, in particular 1,6-GPS-enriched isomalt, in high purity and with a profile of properties optimized for certain areas of application, from isomaltulose and sucrose-containing carbohydrate mixtures, which are preferably obtained enzymatically from sucrose or sucrose-containing starting mixtures.

The present invention is based on the technical problem of overcoming the above disadvantages, in particular a particularly process-stable, cost-effective method for efficiently producing isomalt, in particular one which has a higher 1,6-GPS than 1,1-GPM content, from an isomaltulose- and sucrose-containing carbohydrate mixture obtained, preferably from a sucrose-glucosylmutase-catalyzed conversion of sucrose or sucrose-containing starting mixtures, which is a particularly stable isomalt, in particular for use in sweets, for example hard candies, in high purity of the main components 1,6-GPS and 1,1-GPM and which provides high yield. The invention also provides a particularly pure isomalt provided by means of this method, in particular one that can be referred to as “sugar-free”, in particular a sugar content, in particular sucrose content, of at most 0.50% by weight (based on DM of the isomalt) and at the same time is characterized by particularly low sorbitol and mannitol content.

The present invention solves the technical problem on which it is based by providing the present technical teaching, in particular the teaching of the independent claims and the preferred embodiments disclosed in the description and the dependent claims.

According to the invention, a method for the continuous production of isomalt from an isomaltulose- and sucrose-containing carbohydrate mixture is provided, comprising the method steps
a) Provision of an isomaltulose- and sucrose-containing carbohydrate mixture in an aqueous medium, containing 75.00 to 99.99% by weight isomaltulose and 0.01 to 0.50% by weight sucrose (in each case DM (dry matter), based on the total DM of the carbohydrate mixture), of hydrogen and a ruthenium-based catalyst,
b) Conversion of the carbohydrate mixture to isomalt by continuously bringing the carbohydrate mixture present in the aqueous medium into contact with the ruthenium-based catalyst and hydrogen at a space velocity of 0.25 to 1.5 h⁻¹, at a hydrogen pressure of 16.0 to 22.0 MPa and a pH of 2.0 to 6.0 to obtain an isomalt-containing product stream while setting a reaction temperature of at most 100° C. and
c) Preservation of the isomalt.

The present procedure is accordingly characterized in that in a first method step a) an isomaltulose- and sucrose-containing carbohydrate mixture in an aqueous medium containing 75.00 to 99.99% by weight isomaltulose and 0.01 to 0.50% by weight sucrose, hydrogen and a ruthenium-based catalyst, in particular a supported catalyst, are provided and in a second method step b) the carbohydrate mixture present in the aqueous medium is brought into contact with the ruthenium-based catalyst and hydrogen in a continuous procedure so that a reaction medium is obtained in which the conversion of the carbohydrate mixture to isomalt takes place. According to the invention, the carbohydrate mixture is brought into contact with the ruthenium-based catalyst and hydrogen at a space velocity of 0.25 to 1.5 h⁻¹, at a hydrogen pressure of 16.0 to 22.0 MPa (160 to 220 bar), a pH value of 2.0 to 6.0 and at a temperature of at most 100° C., wherein preferably the isomaltulose is converted in step b) with a conversion rate of 99 to 100 mol % and a selectivity of 97 to 100 mol % to 1,6-GPS (6-O-α-D-glucopyranosyl-D-sorbitol) and 1,1-GPM (1-O-α-D-glucopyranosyl-D-mannitol) to obtain the isomalt in a third method step c).

The procedure according to the invention is particularly and advantageously preferably characterized in that, using the features defined in method steps a) and b), in particular the quantitatively defined isomaltulose and sucrose-containing carbohydrate mixture as starting material and the method parameters specified in method step b), a procedure is provided which leads to a conversion rate of isomaltulose of 99 to 100 mol % and a selectivity of the conversion of isomaltulose to 1,6-GPS and 1,1-GPM of 97 to 100 mol %.

According to the invention, there is therefore preferably provided a method for the continuous production of isomalt from an isomaltulose- and sucrose-containing carbohydrate mixture, comprising the method steps
a) Provision of an isomaltulose and sucrose-containing carbohydrate mixture in an aqueous medium, containing 75.00 to 99.99% by weight isomaltulose and 0.01 to 0.50% by weight sucrose (in each case DM (dry matter), based on the total DM of the carbohydrate mixture), of hydrogen and a ruthenium-based catalyst,
b) Conversion of the carbohydrate mixture to isomalt by continuously bringing the carbohydrate mixture present in the aqueous medium into contact with the ruthenium-based catalyst and hydrogen at a space velocity of 0.25 to 1.5 h⁻¹, at a hydrogen pressure of 16.0 to 22.0 MPa and a pH of 2.0 to 6.0 to obtain an isomalt-containing product stream while setting a reaction temperature of at most 100° C., wherein the isomaltulose in step b) is converted with a conversion rate of 99 to 100 mol % and a selectivity of 97 to 100 mol % to 1,6-GP S (6-O-α-D-glucopyranosyl-D-sorbitol) and 1,1-GPM (1-O-α-D-glucopyranosyl-D-mannitol) and
c) Preservation of the isomalt.

The invention therefore envisages using a carbohydrate mixture containing isomaltulose as the starting material for the method provided according to the invention, the so-called “residual sucrose”, i.e. a comparatively low proportion of sucrose, namely 0.01 to 0.50% by weight sucrose. By setting the method parameters provided according to the invention, when using such a starting material, it is surprisingly possible according to the invention in a preferred embodiment to achieve a product of particularly high purity with the advantages described below. According to the invention it is provided that the starting material defined above, containing isomaltulose with a small proportion of sucrose, is brought into contact at a precisely defined space velocity of 0.25 to 1.5 h⁻¹, at a hydrogen pressure of 16.0 to 22.0 MPa, and a pH of 2.0 to 6.0, with the ruthenium-based catalyst and hydrogen in order to obtain an isomalt-containing product stream, wherein the reaction temperature of this isomalt-containing product stream is at most 100° C. In particular, the reaction temperature of the isomalt-containing product stream is to be set so that an isomaltulose conversion rate of 99 to 100 mol % with a selectivity of 97 to 100 mol % to 1,6-GPS and 1,1-GPM is achieved.

The method conditions used according to the invention are particularly gentle and only lead to a very small extent to the formation of undesirable by-products from the point of view of food law and application technology. In particular, it was surprisingly found that the sucrose present in the carbohydrate mixture used is neither split nor hydrogenated despite the acidic hydrogenation condition. Advantageously, the undesired formation of sorbitol and mannitol from sucrose present in the carbohydrate mixture can therefore be avoided in many applications. Since no sorbitol and mannitol are formed from the sucrose used during the method according to the invention, in cases in which a defined maximum amount of sorbitol and mannitol is desired in the isomalt obtained for reasons of application, for example, the amount of glucose and fructose that may be present in the isomaltulose- and sucrose-containing carbohydrate mixture used for the hydrogenation must be correspondingly larger.

The procedure provided according to the invention of using a maximum temperature of 100° C. in conjunction with the further features of the teaching according to the invention during the conversion of the carbohydrate mixture to isomalt reduces energy costs and provides advantageous isomalt efficiently and quickly in a cost-effective method.

In a preferred embodiment, the method according to the invention can produce an isomalt which complies with the JECFA specification and is also referred to below as a JECFA-compliant isomalt. Such an isomalt is characterized by a content of at least 98% by weight hydrogenated mono- and disaccharides, namely 1,6-GPS, 1,1-GPM, 1,1-GPS, sorbitol and mannitol, and a maximum 2% by weight secondary components, wherein this isomalt has at least 86% by weight 1,6-GPS and 1,1-GPM and 0 to 0.3% by weight reducing sugars (for example glucose and fructose), and wherein in this isomalt at most 0.5% by weight, in particular 0.01 to 0.50% by weight, in particular 0.01 to 0.49% by weight, in particular 0.01 to 0.05% by weight sucrose (in each case based on the total weight of the dry matter of the isomalt) are available.

In a particularly preferred embodiment, the method according to the invention can also produce an isomalt which is a particularly pure isomalt, also referred to here as a highly pure isomalt. The highly pure isomalt produced according to the invention in a preferred embodiment corresponds to the Joint FAO/WHO Expert Committee on Food Additives Specification (69th JECFA (2008), published in FAO JECFA Monographs 5 (2008)) and is accordingly a JECFA-compliant isomalt with a content of at least 98.00% by weight hydrogenated mono- and disaccharides, namely 1,6-GPS, 1,1-GPM, 1,1-GPS, sorbitol and mannitol, and at most 2.00% by weight secondary components, wherein at least 98.00% by weight 1,6-GPS and 1,1-GPM, 0 to 0.50% by weight sorbitol, 0 to 0.50% by weight mannitol and 0 to 0.30% by weight reducing sugars (for example glucose and fructose) are present, wherein some of the 2% by weight secondary components possibly present in this isomalt are each present in an amount of 0 to 0.50% by weight and a total of at most 0.50% by weight reducing and non-reducing sugars (such as isomaltulose, isomaltose, sucrose, glucose or fructose), and at most 0.5% by weight, in particular 0.01 to 0.50% by weight, in particular 0.01 to 0.49% by weight, in particular 0.01 to 0.05% by weight sucrose, (each based on the total weight of the dry matter of the isomalt) are available. Such an isomalt, also referred to here as highly pure isomalt, is preferably and advantageously characterized in particular by high purity due to a particularly high proportion of the main isomalt components 1,1-GPM and 1,6-GPS, a low but present proportion of sucrose with regard to byproducts and by a low degree of hygroscopicity, in particular a low sorbitol and mannitol content.

In a preferred embodiment of the invention, in particular the formation of at least one, preferably all of the substances or substance classes (byproducts) mentioned below, is statistically significantly reduced compared to isomalt not obtained according to the invention: trisaccharide alcohols, non-reducing trisaccharides, glucosylglycerols, glucosyl tetraols, glucosyl pentitols, further glucosyl glycitols, deoxydisaccharide alcohols, dideoxydisaccharide alcohols, glycerol, tetraols (for example erythritol or threitol), pentitols (for example ribitol (adonite), arabitol, xylitol or lyxitol), deoxyhexitols, dideoxyhexitols, sorbitol, mannitol, galactitol, allitol, gulitol, iditol, altritol, or talitol.

The teaching of the present invention provides, in a surprising way, a particularly selective, efficient and gentle method for the production of isomalt from a carbohydrate mixture containing sucrose and isomaltulose or, in one embodiment, sucrose, isomaltulose and trehalulose, in which the sucrose under acidic conditions during a ruthenium-based hydrogenation, contrary to the expectations from the prior art, for example EP 2 361 255 B 1, is not split hydrogenated, but remains chemically unchanged and is not split. There is therefore no formation of sorbitol and mannitol through sucrose splitting and hydrogenation. The procedure according to the invention thus provides for avoiding the splitting hydrogenation of sucrose to sorbitol and mannitol, in particular sorbitol, during the conversion of the sucrose-containing carbohydrate mixture containing isomaltulose or isomaltulose and trehalulose with hydrogen on a ruthenium catalyst to yield isomalt, and thus to provide an isomalt which has excellent properties for further processing in, for example, sweets such as hard candies, lozenges, chocolate, chewing gum, ice cream or in baked goods or in medicines. The inventors have surprisingly found that the sucrose present in small amounts in the starting material, i.e. the carbohydrate mixture, is not split under the conditions according to the invention, i.e. in particular pressure, temperature, pH, and space velocity. The “residual sucrose”, which is usually contained in the “isomerized sucrose” obtained by glucosylmutase-catalyzed conversion of sucrose or sucrose-containing starting mixtures, advantageously does not contribute to an increase in the sorbitol and mannitol content in the isomalt obtained according to the invention because it is not split and not hydrogenated, so that, if necessary, higher glucose and fructose contents can also be used in the isomaltulose and sucrose-containing carbohydrate mixture provided in method step a).

The procedure according to the invention is also technologically advantageous in that a significantly more moderate temperature is required for isomaltulose hydrogenation than for simultaneous splitting hydrogenation of sucrose, since a significantly higher temperature is necessary for hydrogenation of the glucose resulting from the splitting of sucrose in addition to fructose. In addition, complex and costly drying steps are reduced or avoided, which would result from the hygroscopicity of sorbitol and/or other by-products formed from the splitting hydrogenation of sucrose and thus a simple, process-stable and cost-conscious production method for isomalt, in particular a 1,6-GPS enriched isomalt, is made possible. The low proportion of sucrose in the isomalt produced is advantageous in that the isomalt produced is suitable for diabetics compared to pure sucrose, is gentle on the teeth, and has a lower calorific value. The sweetener produced according to the invention is characterized in particular by increased storage stability, in particular in humid and hot climatic regions.

In connection with the present invention, “bringing into contact” means that the aqueous medium, in particular an aqueous solution, is brought into physical contact with a catalyst and hydrogen while supplying hydrogen, in particular that the medium, in particular the solution, flows past the catalyst, in particular flows through a catalyst bed containing the catalyst. Without being bound by theory, the catalyst accelerates the conversion of isomaltulose or isomaltulose and trehalulose in the carbohydrate mixture, containing isomaltulose or isomaltulose and trehalulose and sucrose, with hydrogen.

Bringing the carbohydrate mixture into contact with the ruthenium-based catalyst while supplying hydrogen leads to a conversion of the carbohydrate mixture to isomalt. According to the invention, it is provided that bringing the carbohydrate mixture into contact with the ruthenium-based catalyst and hydrogen represents a hydrogenation of the carbohydrate mixture.

In connection with the present invention, the term “conversion of a carbohydrate mixture to isomalt” is understood to mean that the isomaltulose present in the carbohydrate mixture is partially or completely converted to 1,6-GPS and 1,1-GPM with the aid of hydrogen, i.e. is hydrogenated. If the carbohydrate mixture containing isomaltulose and sucrose contains further hydrogenatable constituents, for example trehalulose, it can be provided that these are also converted, i.e. hydrogenated, in particular trehalulose to 1,1-GPS and 1,1-GPM, during the bringing into contact with the ruthenium-based catalyst. According to the invention, the sucrose, in particular residual sucrose, present in the carbohydrate mixture containing isomaltulose or isomaltulose and trehalulose as well as sucrose, does not react during method step b). Splitting and/or hydrogenation of sucrose is avoided according to the invention.

In connection with the present invention, the “reaction medium” obtained according to method step b) is understood to mean a medium which is formed when the carbohydrate mixture present in the aqueous medium is continuously brought into contact under the influence of a ruthenium-based catalyst and with the addition of hydrogen, wherein this is in the preferred embodiment comprises the components isomaltulose or isomaltulose and trehalulose and sucrose as well as the products formed during the reaction, in particular 1,6-GPS and 1,1-GPM, provided in method step a).

In connection with the present invention, the term “space velocity” is understood to mean the quotient of the volume flow of the carbohydrate mixture present in the aqueous medium and the volume of the ruthenium-based catalyst velocity_{space}=V (liquid volume)/V (catalyst volume) per hour (m³/h×m³=1/h, also referred to as LHSV: liquid hourly space velocity). The volume of the catalyst relates to the macroscopic volume of the catalyst, regardless of the shape or structure of the catalyst.

In connection with the present invention, “continuously” means that the carbohydrate mixture present in the aqueous medium is brought into contact with the catalyst at a space velocity which is constantly greater than 0 h⁻¹.

In connection with the present invention, isomalt is understood to mean a sugar substitute which comprises 1,6-GPS and 1,1-GPM as the main component, in particular at least 86% by weight 1,6-GP S and 1,1-GPM.

A “1,6-GPS-enriched isomalt” is an isomalt with a proportion of 1,6-GPS that is greater than the 1,1-GPM proportion, i.e. a 1,6-GP S to 1,1-GPM ratio of >1 (based on DM content of 1,6-GPS and 1,1-GPM in isomalt).

In a preferred embodiment, the term isomalt is understood to mean a JECFA-compliant isomalt. In a particularly preferred embodiment, the term isomalt is understood to mean a highly pure isomalt.

In connection with the present invention, a “JECFA-compliant isomalt” is understood to mean an isomalt which contains at least 98% by weight of hydrogenated mono- and disaccharides, namely 1,6-GPS, 1,1-GPM, 1,1-GPS, sorbitol and mannitol, and at most 2% by weight secondary components, wherein at least 86% by weight 1,6-GPS and 1,1-GPM, 0 to 0.3% by weight reducing sugars and at most 0.50% by weight, in particular 0.01 to 0.50% by weight sucrose (each based on the total weight of the dry matter of the isomalt) are present.

In connection with the present invention, a “highly pure isomalt” is understood to mean an isomalt which contains at least 98% by weight of hydrogenated mono- and disaccharides, namely 1,6-GPS, 1,1-GPM, 1,1-GPS, sorbitol and mannitol, and at most 2.00% by weight secondary components, wherein at least 98.00% by weight 1,6-GPS and 1,1-GPM, 0 to 0.50% by weight of sorbitol, 0 to 0.50% by weight mannitol, 0 to 0.30% by weight reducing sugars, some of the aforementioned 2% by weight secondary components, if present, in an amount of 0 to 0.50% by weight each and a total of at most 0.50% by weight reducing and non-reducing sugars and at most 0.50% by weight, in particular 0.01 to 0.50% by weight of sucrose (each based on the total weight of the dry matter of the isomalt) are present in the isomalt.

In connection with the present invention, the term “at least 98.00% by weight hydrogenated mono- and disaccharides” is understood to mean the amount of hydrogenated mono- and disaccharides in the isomalt composition comprising at least 98.00% by weight, which are selected from the group consisting of 1,6-GPS, 1,1-GPM, 1,1-GPS, mannitol and sorbitol.

In connection with the present invention, the term “at most 2.0% by weight secondary components” is understood to mean all substances present in an isomalt composition that do not contain any hydrogenated mono- or disaccharides are selected from the group consisting of 1,6-GP S, 1,1-GPM, 1,1-GPS, mannitol and sorbitol.

In connection with the present invention, “individual secondary components” are individual substances that represent the secondary components in their entirety and wherein these individual substances, for example isomaltose, sucrose, glucose, fructose, isomaltulose, glycerol, glucopyranosylidite, isomelezitose, are each individual substances belonging to the substance groups monosaccharides, disaccharides, deoxydisaccharide alcohols, trisaccharides, glucosylglycerols, glucosyltetritols, glucosylpentitols, trisaccharide alcohols, glycosylated disaccharide alcohols or hydrogenated oligomers.

The contents of isomaltulose, trehalulose and/or sucrose in the carbohydrate mixture provided and determined according to the invention and the contents of 1,6-GPS (6-O-α-D-glucopyranosyl-D-sorbitol), 1,1-GPM (1-O-α-D-glucopyranosyl-D-mannitol), sucrose, 1,1-GPS (α-D-glucopyranosyl-1,1-D-sorbitol) and/or isomaltulose and any other components present in the isomalt are preferred by means of GC-FID (GC flame ionization detector) or GC mass spectrometry, particularly preferably using GC-FID with a limit of quantification of 0.01 g/100 g DM with a signal/noise ratio of at least 10:1 according to FCC General Information/Validation, United States Pharmacopeia and JECFA (1996), FNP52, Add/4 (Joint FAO/WHO Expert Committee on Food Additives).

In connection with the present invention, unless otherwise indicated and/or recognizable, the percentages of individual components indicated for a composition of components add up to 100% by weight of the composition in connection with the respectively indicated percentage ranges.

If, in connection with the present invention, the first and second decimal places or the second decimal place are/is not specified in a number, these are/is to be set to 0.

If, in connection with the present invention, a “presence”, “containing” or “comprising” a component in an amount of 0% by weight is mentioned, this means that the respective component is not present in a measurable amount, in particular is not present.

Unless otherwise stated, in connection with the present invention the term “carbohydrate mixture” is understood to mean the carbohydrate mixture according to method step a), that is to say a mixture containing isomaltulose and sucrose or containing isomaltulose, trehalulose and sucrose.

The carbohydrate mixture used according to the invention comprises isomaltulose and sucrose, in particular consists of these.

In a preferred embodiment, the carbohydrate mixture comprises isomaltulose, trehalulose and sucrose, in particular consists of these.

In a particularly preferred embodiment, the carbohydrate mixture comprises isomaltulose and sucrose and at least one further substance, in particular selected from the group consisting of fructose, glucose, isomaltose, trehalulose and oligomers of carbohydrates, in particular consists of these. In a particularly preferred embodiment, the carbohydrate mixture comprises isomaltulose, trehalulose and sucrose and at least one further substance, in particular selected from the group consisting of fructose, glucose, isomaltose and oligomers of carbohydrates, in particular consists of these.

In connection with the present invention, “oligomers of carbohydrates” are understood to mean oligomers and/or polymers of monosaccharides having at least three monosaccharide units 3 and having a homogeneous or heterogeneous monosaccharide composition.

The carbohydrate mixture preferably comprises isomaltulose and sucrose and a substance selected from the group consisting of trehalulose and isomaltose, in particular consists of these. In a particularly preferred embodiment of the present invention, the isomaltulose and sucrose-containing carbohydrate mixture in addition to sucrose and isomaltulose or in addition to sucrose, isomaltulose and trehalulose, additionally glucose, fructose and isomaltose, in particular consists of these, optionally together with oligomers of carbohydrates.

In a particularly preferred embodiment of the present invention, the carbohydrate mixture comprises a maximum of 0.50% by weight, in particular a maximum of 0.40% by weight, in particular a maximum of 0.30% by weight, in particular a maximum of 0.20% by weight (in each case DM based on total dry matter of the carbohydrate mixture) glucose, fructose, oligomers of carbohydrates and/or isomaltose.

In a particularly preferred embodiment of the present invention, the carbohydrate mixture comprises a maximum of 2.00% by weight, in particular a maximum of 1.00% by weight, in particular a maximum of 0.50% by weight, in particular a maximum of 0.40% by weight (each DM based on total dry matter of the carbohydrate mixture) of a total amount of glucose, fructose, oligomers of carbohydrates and/or isomaltose.

In a particularly preferred embodiment of the present invention, the carbohydrate mixture comprises at most 0.50% by weight, in particular at most 0.40% by weight, in particular at most 0.30% by weight, in particular at most 0.20% by weight, in particular at most 0.10% by weight (in each case DM based on total dry matter of the carbohydrate mixture) glucose.

In a particularly preferred embodiment of the present invention, the carbohydrate mixture comprises at most 0.50% by weight, in particular at most 0.40% by weight, in particular at most 0.30% by weight, in particular at most 0.20% by weight, in particular at most 0.10% by weight (in each case DM based on total dry matter of the carbohydrate mixture) fructose.

In a particularly preferred embodiment of the present invention, the carbohydrate mixture comprises at most 0.50% by weight, in particular at most 0.40% by weight, in particular at most 0.30% by weight, in particular at most 0.20% by weight, in particular at most 0.10% by weight (in each case DM based on total dry matter of the carbohydrate mixture) isomaltose.

In a particularly preferred embodiment of the present invention, the carbohydrate mixture comprises at most 0.50% by weight, in particular at most 0.40% by weight, in particular at most 0.30% by weight, in particular at most 0.20% by weight, in particular at most 0.10% by weight (in each case DM based on total dry matter of the carbohydrate mixture) oligomers of carbohydrates.

In a particularly preferred embodiment of the present invention, the carbohydrate mixture provided in method step a) comprises no glucose or no fructose or no isomaltose, in particular no glucose and no fructose and no isomaltose, in particular no glucose and no fructose, in particular no glucose, in particular no fructose.

In a particularly preferred embodiment, the carbohydrate mixture provided in method step a) does not have any oligomers of carbohydrates.

In a particularly preferred embodiment of the present invention, the carbohydrate mixture prepared in method step a), containing isomaltulose or isomaltulose and trehalulose and sucrose, is a carbohydrate mixture obtained by reacting sucrose or sucrose-containing starting mixtures, in particular in an aqueous medium, in particular aqueous solution, with sucrose glucosyl mutases.

In a particularly preferred embodiment of the present invention, the carbohydrate mixture provided in method step a) is a so-called “isomerized sucrose,” in particular one which was produced enzymatically from sucrose or sucrose-containing starting mixtures, in particular as described in EP 0 625 578 A1.

With regard to the production of “isomerized sucrose” and the means for its production, the disclosure content in the patent application mentioned is fully incorporated into the disclosure content of the present teaching.

The enzymatic conversion of sucrose or sucrose-containing mixtures to the carbohydrate mixture provided according to method step a), which is preferably provided according to the invention, is preferably an enzymatic conversion by means of a sucrose glucosyl mutase. The reaction can preferably take place by means of a sucrose-glucosylmutase by using bacteria comprising sucrose-glucosylmutase, in particular selected from the group consisting of Protaminobacter rubrum, Serratia plymuthica, Serratia marcescens, Erwinia rhapontici, Leuconostoc mesenteroides, Pseudomonas mesoacidophila, Agrobacterium radiobacter and combinations thereof.

In a preferred embodiment, the invention therefore relates to a method in which the isomaltulose and sucrose-containing carbohydrate mixture provided in method step a) was obtained by enzymatic reaction of sucrose or a sucrose-containing starting mixture with a sucrose-glucosylmutase.

The carbohydrate mixture, preferably obtained from sucrose or sucrose-containing starting mixture by means of sucrose-glucosylmutases, can either be provided directly according to method step a) and then reacted immediately according to method step b) or, in a particularly preferred embodiment, it can be converted prior to provision according to method step a), in a method step a0) to reduce the sucrose content. The method step for reducing the sucrose content optionally provided in method step a0) is particularly necessary if the carbohydrate mixture provided in method step a) is to be obtained from a source, for example an “isomerized sucrose” obtained through enzymatic reaction of sucrose or a sucrose-containing starting mixture with a sucrose-glucosylmutase, in which there is a higher sucrose content and accordingly the sucrose content must be reduced to such an extent that a carbohydrate mixture with the sucrose content according to method step a) is obtained.

In a particularly preferred embodiment of the present invention, the carbohydrate mixture provided in method step a) is a carbohydrate mixture obtained by enzymatic reaction of sucrose or a sucrose-containing starting mixture with a sucrose-glucosylmutase and is carried out in a method step a0) to reduce the sucrose content to a content of 0.01 to 0.50% by weight sucrose (dry matter based on total dry matter of the carbohydrate mixture).

In a particularly preferred embodiment of the present invention, method step a0) is a crystallization for partial separation of sucrose, a chromatography on ion exchangers for partial separation of sucrose, an enzymatic splitting of sucrose, for example by means of invertase, while maintaining a residual amount of sucrose from 0.01 to 0.5% by weight or a combination of these methods.

In a particularly preferred embodiment of the present invention, the carbohydrate mixture provided in method step a) contains 0.01 to 0.45% by weight, in particular 0.01 to 0.40% by weight, in particular 0.01 to 0.35% by weight, in particular 0.01 to 0.30% by weight, in particular 0.01 to 0.25% by weight, in particular 0.01 to 0.20% by weight, in particular 0.01 to 0.15% by weight, in particular 0.01 to 0.10% by weight, in particular 0.01 to 0.09% by weight, in particular 0.01 to 0.08% by weight, in particular 0, 01 to 0.07% by weight, in particular 0.01 to 0.06% by weight, in particular 0.01 to 0.05% by weight, in particular 0.01 to 0.04% by weight, in particular 0.01 to 0.03% by weight, in particular 0.01 to 0.02% by weight, in particular 0.02 to 0.50% by weight, in particular 0.02 to 0.45% by weight, in particular 0.02 to 0.40% by weight, in particular 0.02 to 0.30% by weight, in particular 0.02 to 0.20% by weight, in particular 0.02 to 0.10% by weight, in particular 0.02 to 0.08% by weight, in particular 0.02 to 0.06% by weight, in particular 0.02 to 0.05% by weight, in particular 0.02 to 0.04% by weight, sucrose (in each case based on the dry matter of the carbohydrate mixture).

The sucrose content in the carbohydrate mixture provided in method step a) is preferably from 0.01 to 0.05% by weight (based on the dry matter of the carbohydrate mixture).

In a particularly preferred embodiment of the present invention, the carbohydrate mixture provided in method step a) contains 76.00 to 99.99% by weight isomaltulose, in particular 78.00 to 99.99% by weight, in particular 80.00 to 99.99% by weight, in particular 84 to 99.99% by weight, in particular 86.00 to 99.99% by weight, in particular 90.00 to 99.99% by weight, in particular 92.00 to 99.99% by weight, in particular 94.00 to 99.99% by weight, in particular 96.00 to 99.99% by weight, in particular 98.00 to 99.99% by weight, in particular 76.00 to 99.80% by weight, in particular 78.00 to 99.80% by weight, in particular 80.00 to 99.80% by weight, in particular 84.00 to 99.80% by weight, in particular 86.00 to 99.80% by weight, in particular 90.00 to 99.80% by weight, in particular 92.00 to 99.80% by weight, in particular 94.00 to 99.80% by weight, in particular 96.00 to 99.80% by weight, in particular 98.00 to 99.80% by weight, in particular 76.00 to 99.50% by weight, in particular 78.00 to 99.50% by weight %, in particular 80.00 to 99.50% by weight, in particular 84.00 to 99.50% by weight, in particular 86 to 99.50% by weight, in particular 90.00 to 99.50% by weight, in particular 92.00 to 99.50% by weight, in particular 94.00 to 99.50% by weight, in particular 95.00 to 99.50% by weight, in particular 96.00 to 99.50% by weight, in particular 97.00 to 99.50% by weight %, in particular 98.00 to 99.50% by weight, in particular 97.70 to 99.30% by weight isomaltulose (in each case based on the dry matter of the carbohydrate mixture).

In a particularly preferred embodiment, the carbohydrate mixture provided in method step a) preferably has an isomaltulose content of 86.00 to 99.99% by weight, in particular 90.00 to 99.99% by weight, in particular 95.00 to 99.99% by weight, in particular 96.00 to 99.99% by weight, in particular 97.00 to 99.99% by weight, in particular 98.00 to 99.99% by weight, in particular 98.50 to 99.99% by weight, in particular 98.60 to 99.99% by weight (in each case based on the dry matter of the carbohydrate mixture).

The isomaltulose content of the carbohydrate mixture provided in method step a) is preferably 86.00 to 99.99% by weight isomaltulose (based on the dry matter of the carbohydrate mixture).

The isomaltulose content in the carbohydrate mixture provided in method step a) is preferably 98.00 to 99.80% by weight of isomaltulose (based on the dry matter of the carbohydrate mixture).

In a particularly preferred embodiment of the present invention, the carbohydrate mixture comprises trehalulose.

In a particularly preferred embodiment of the present invention, the 75.00 to 99.99% by weight isomaltulose and 0.01 to 0.50% by weight sucrose-containing carbohydrate mixture provided in method step a) contains 75.01 to 100.00% by weight, in particular 80.00 to 95.00% by weight, in particular 86.00 to 90.00% by weight, in particular 90.00 to 99.00% by weight, in particular 99.00 to 100.00% by weight, in particular 75.01 to 99.99% by weight, in particular 80.00 to 99.99% by weight, in particular 90.00 to 99.99% by weight, in particular 92.00 up to 99.99% by weight, in particular 94.00 to 99.99% by weight, in particular 96.00 to 99.99% by weight, in particular 98.00 to 99.99% by weight, in particular 99.00 to 99.99% by weight, in particular 75.01 to 99.80% by weight, in particular 80.00 to 99.80% by weight, in particular 85.00 to 99.80% by weight, in particular 90.00 to 99.80% by weight, in particular 98.00 to 99.80% by weight, in particular 98.50 to 99.80% by weight, in particular 98.60 to 99.80% by weight, in particular 98.70 to 99.80% by weight, in particular 98.80 to 99.80 by weight, in particular 98.90 to 99.80% by weight, in particular 99.00 to 99.80% by weight, in particular 99.10 to 99.80% by weight isomaltulose and sucrose, wherein optionally, adding up to 100% by weight of the carbohydrate mixture, trehalulose, glucose, fructose, isomaltose and/or oligomers of carbohydrates are present (each based on the dry matter of the carbohydrate mixture).

The sucrose and isomaltulose content in the carbohydrate mixture provided in method step a) is preferably 98.00 to 99.99% by weight, wherein optionally, adding up to 100% by weight of the carbohydrate mixture, trehalulose, glucose, fructose, isomaltose and/or oligomers of carbohydrates are present (based on the dry matter of the carbohydrate mixture).

The sucrose, isomaltulose and trehalulose content in the carbohydrate mixture provided in method step a) is preferably 98.00 to 99.80% by weight (based on the dry matter of the carbohydrate mixture), wherein the isomaltulose content is 97.70 to 99.30% by weight, the trehalulose content is 0.29 to 1.00% by weight, and the sucrose content is 0.01 to 0.05% by weight (each based on the dry matter of the carbohydrate mixture).

The sucrose, isomaltulose and trehalulose content in the carbohydrate mixture provided in method step a) is preferably 98.00 to 99.80% by weight (based on the dry matter of the carbohydrate mixture), wherein the isomaltulose content is 97.70 to 99.30% by weight, the trehalulose content is 0.29 to 1.00% by weight and the sucrose content is 0.01 to 0.05% by weight (each based on the dry matter of the carbohydrate mixture), and wherein, adding up to 100% by weight of the carbohydrate mixture, glucose, fructose, isomaltose and/or oligomers of carbohydrates are present (based on the dry matter of the carbohydrate mixture).

The sucrose, isomaltulose and trehalulose content in the carbohydrate mixture provided in method step a) is preferably 98 to 99.80% by weight (based on the dry matter of the carbohydrate mixture), wherein the isomaltulose content is 98.00 to 99.30% by weight, the trehalulose content is 0.29 to 1.0% by weight, and the sucrose content is 0.01 to 0.05% by weight (each based on the dry matter of the carbohydrate mixture).

The sucrose, isomaltulose and trehalulose content in the carbohydrate mixture provided in method step a) is preferably 98 to 99.80% by weight (based on the dry matter of the carbohydrate mixture), wherein the isomaltulose content is 98.00 to 99.30% by weight, the trehalulose content is 0.29 to 1.0% by weight and the sucrose content is 0.01 to 0.05% by weight (each based on the dry matter of the carbohydrate mixture), and wherein, adding up to 100% by weight of the carbohydrate mixture, glucose, fructose, isomaltose and/or oligomers of carbohydrates are present (based on the dry matter of the carbohydrate mixture).

In a particularly preferred embodiment of the present invention, the isomaltulose- and sucrose-containing carbohydrate mixture provided in method step a) comprises 0.01 to 24.99% by weight trehalulose, in particular 0.01 to 24.50% by weight, in particular 0.01 to 21.00% by weight, in particular 0.01 to 20.00% by weight, in particular 0.01 to 19.00% by weight, in particular 0.01 to 18.00% by weight, in particular 0.01 to 17.00% by weight, in particular 0.01 to 10.00% by weight, in particular 0.01 to 5.00% by weight, particularly preferably 0.50 to 25.00% trehalulose, in particular 0.50 to 23.00% by weight, in particular 0.50 to 21.00% by weight, in particular 0.50 to 20.00% by weight, in particular 0.50 to 19.00% by weight, in particular 0.50 to 18.00% by weight, in particular 0.50 to 17.00% by weight, in particular 0.50 to 10.00% by weight, in particular 0.50 to 5.00% by weight, particularly preferably 0.30 to 1.00% by weight, particularly preferably 0.29 to 1.00% by weight, particularly preferably 1.00 to 25.00% by weight, in particular 1.00 to 23.00% by weight, in particular 1.00 to 21.00% by weight, in particular 1.00 to 20.00% by weight, in particular 1.00 to 10.00% by weight, in particular 5.00 up to 25.00% by weight, in particular 5.00 to 23.00% by weight, in particular 5.00 to 22.00% by weight, in particular 5.00 to 20.00% by weight, in particular 5.00 to 10.00% by weight, in particular 10.00 to 25.00% by weight, in particular 10.00 to 22.00% by weight, in particular 10.00 to 20.00% by weight, in particular 10.00 to 18.00% by weight trehalulose (in each case based on the dry matter of the carbohydrate mixture).

The carbohydrate mixture preferably has 0.30 to 1.00% by weight trehalulose (based on the dry matter of the carbohydrate mixture).

If, in a further particularly preferred embodiment, the carbohydrate mixture also contains trehalulose in addition to sucrose and isomaltulose, the carbohydrate mixture preferably contains 75.00 to 99.49% by weight isomaltulose and 0.50 to 24.99% by weight trehalulose, in particular 75.00 to 86.00% by weight isomaltulose and 13.99 to 24.99% by weight trehalulose (each based on the dry matter of the carbohydrate mixture).

In a particularly preferred embodiment of the present invention, the ratio of isomaltulose to trehalulose in the carbohydrate mixture (% by weight in DM) is 98.00 to 99.50, is in particular 98.50 to 99.40 parts of isomaltulose to 0.50 to 1.50, in particular 0.60 to 1.20 parts of trehalulose.

In a preferred embodiment, the ratio of isomaltulose to trehalulose (% by weight in DM) in the carbohydrate mixture is 3 to 1, in particular 4 to 1, in particular 10 to 1, in particular 85 to 1, in particular 150 to 1, in particular 1,500 to 1, in particular 9,998 to 1.

In a particularly preferred embodiment, the aqueous medium provided in method step a) is an aqueous solution, an aqueous suspension, an aqueous syrup or an aqueous colloidal composition.

In a preferred embodiment of the present invention, the dry matter content of the carbohydrate mixture present in an aqueous medium, in particular in an aqueous solution, is 10.00 to 60.00% by weight (based on the total weight of the medium).

In a particularly preferred embodiment of the present invention, the dry matter content of the carbohydrate mixture present in aqueous solution is 15.00 to 60.00% by weight, in particular 20.00 to 60.00% by weight, in particular 30.00 to 60.00% by weight, in particular 35.00 to 60.00% by weight, in particular 40.00 to 60.00% by weight, in particular 45.00 to 60.00% by weight, in particular 50.00 to 60.00% by weight, in particular 10.00 to 55.00% by weight, in particular 10.00 to 50.00% by weight, in particular 10.00 to 45.00% by weight, in particular 10.00 up to 40% by weight, in particular 10.00 to 35.00% by weight, in particular 10.00 to 30.00% by weight, in particular 10.00 to 25.00% by weight, in particular 10.00 up to 20.00% by weight, in particular 10.00 to 15.00% by weight, in particular 20.00 to 50.00% by weight, in particular 20.00 to 45.00% by weight, in particular 20.00 to 30.00% by weight, in particular 30.00 to 50.00% by weight, in particular 35.00 to 50.00% by weight, in particular 35.00 to 45.00% by weight (each based on total weight of the medium, in particular the solution).

The dry matter content of the carbohydrate mixture present in aqueous solution is preferably 35.00 to 45.00% by weight, based on the total weight of the medium.

In a particularly preferred embodiment, it is provided that the carbohydrate mixture provided in method step a) is heated, that is to say preheated, in a method step a1) before the reaction provided in method step b).

In a particularly preferred embodiment, the carbohydrate mixture provided in method step a), in particular after carrying out an optional method step a0) which reduces the sucrose content, is preheated to a temperature prior to the reaction provided in method step b) which at most corresponds to the reaction temperature according to method step b), in particular to a temperature which corresponds to the reaction temperature in method step b).

In a particularly preferred embodiment, in a method step a1) the isomaltulose- and sucrose-containing carbohydrate mixture provided in method step a), in particular after carrying out an optional method step a0) which reduces the sucrose content, is heated to a temperature of 30 to 80° C., in particular 30 to 75° C., in particular 30 to 70° C., in particular 45 to 68° C., in particular 46 to 67.5° C.

In a particularly preferred embodiment, the isomaltulose- and sucrose-containing carbohydrate mixture is preheated to a temperature of 30 to 80° C. in a method step a1) which is provided in method step a), in particular after carrying out an optional method step a0) which reduces the sucrose content.

In a particularly preferred embodiment, the isomaltulose- and sucrose-containing carbohydrate mixture provided in method step a) is preheated to a temperature of 30 to 75° C. in a method step a1), in particular after carrying out an optional method step a0) which reduces the sucrose content.

In a particularly preferred embodiment, the isomaltulose- and sucrose-containing carbohydrate mixture provided in method step a) is preheated to a temperature of 30 to 70° C. in a method step a1), in particular after carrying out an optional method step a0) which reduces the sucrose content.

The carbohydrate mixture provided in method step a) or the carbohydrate mixture preheated in the optional form according to method step a1) is then fed to method step b), in particular fed into a reactor suitable for the reaction provided in method step b).

In a preferred embodiment, the continuous method provided according to method step b) is carried out in a fixed-bed reactor.

In a preferred embodiment, the continuous method provided according to method step b) is carried out as a trickle-bed procedure, preferably in a trickle-bed reactor.

In a preferred embodiment, the continuous procedure can also be carried out in a fixed bed reactor which is operated as a bubble reactor.

In a preferred embodiment, the continuous procedure can also be carried out in a continuous stirred tank (CSTR) or in a stirred tank cascade.

In a particularly preferred embodiment of the invention, the hydrogen pressure in method step b) is 16.2 to 22.0 MPa, in particular 16.5 to 22.0 MPa, in particular 16.5 to 21.5 MPa, in particular 16.5 to 21.0 MPa, in particular 16.5 to 20.0 MPa, in particular 16.5 to 19.5 MPa, in particular 16.5 to 19.0 MPa, in particular 16.5 to 18.5 MPa, in particular 16.5 to 18.0 MPa.

In a preferred embodiment, the hydrogen pressure in method step b) is 17.0 to 22.0 MPa, in particular 17.0 to 21.0 MPa, in particular 17.0 to 20.0 MPa, in particular 17.0 to 19.5 MPa, in particular 17.0 to 19.0 MPa, in particular 17.0 to 18.5 MPa, in particular 17.5 to 22.0 MPa, in particular 17.5 to 19.0 MPa, in particular 17.5 to 18.5 MPa, in particular 17.5 to 18.0 MPa, in particular 18.0 MPa.

The hydrogen pressure in method step b) is preferably 16.5 to 21.0 MPa.

In a particularly preferred embodiment, the hydrogen pressure in method step b) is 16.00 to 19.00 MPa, in particular 16.00 to 17.00 MPa, in particular 16.5 MPa.

In a particularly preferred embodiment of the present invention, the space velocity in method step b) is 0.27 to 1.5 h⁻¹, in particular 0.3 to 1.5 h⁻¹, in particular 0.3 to 1.0 h⁻¹, in particular 0.3 to 0.9 h⁻¹, in particular 0.3 to 0.8 h⁻¹, in particular 0.3 to 0.7 h⁻¹, in particular 0.3 to 0.6 h⁻¹, in particular 0.3 to 0.5 h⁻¹, in particular 0.4 to 1.5 h⁻¹, in particular 0.4 to 1.25 h⁻¹, in particular 0.4 to 1.0 h⁻¹, in particular 0.4 up to 0.8 h⁻¹, in particular 0.4 to 0.7 h⁻¹, in particular 0.4 to 0.6 h⁻¹, in particular 0.25 to 1.0 h⁻¹, in particular 0.25 to 0.9 h⁻¹, in particular 0.25 to 0.8 h⁻¹, in particular 0.25 to 0.7 h⁻¹, in particular 0.25 to 0.6 h⁻¹, in particular 0.25 to 0.5 h⁻¹, in particular 0.45 to 1.5 h⁻¹, in particular 0.46 to 1.5 h⁻¹, in particular 0.48 to 1.5 h⁻¹, in particular 0.48 to 1.0 h⁻¹, in particular exactly 0.3 h⁻¹, in particular exactly 0.4 h⁻¹, in particular exactly 0.5 h⁻¹, in particular exactly 0.7 h⁻¹, in particular exactly 1.0 h⁻¹.

In a particularly preferred embodiment of the present invention, the space velocity in method step b) is 0.25 to 0.9 h⁻¹.

In a particularly preferred embodiment of the present invention, the space velocity in method step b) is 0.3 to 0.9 h⁻¹.

In a particularly preferred embodiment of the present invention, the pH in method step b) is 2.0 to 5.5, in particular 2.0 to 5.4, in particular 2.0 to 5.3, in particular 2.0 to 5.0, in particular 2.0 to 4.0, in particular 2.5 to 6.0, in particular 2.5 to 5.8, in particular 2.5 to 5.5, in particular 2.5 to 4.0, in particular 2.7 to 3.3, in particular 2.8 to 3.0, in particular 3.0 to 6.0, in particular 3.0 to 5.5, in particular 3.0 to 5.0, in particular 3.0 to 4.5, in particular 3.0 to 4.0, in particular 3.0 to 4.0, in particular 4.0 to 6.0, in particular 4.0 to 5.5, in particular 4.0 to 5.0, in particular 4.0 to 4.5, in particular 4.0 to 4.0, in particular 4.0 to 6.0, in particular 4.0 to 5.5, in particular 4.0 to 5.0, in particular 4.0 to 4.5, in particular 4.5 to 6.0, in particular 4.5 to 5.5, in particular 4.5 to 5.0, in particular 5.0 to 6.0.

In a particularly preferred embodiment, the pH in method step b) is 2.5 to 6.0, in particular 2.5 to 5.8, in particular 2.5 to 5.5.

In a particularly preferred embodiment of the present invention, the pH in method step b) is 2.5 to 5.9.

In a particularly preferred embodiment of the present invention, the pH in method step b) is 3.4 to 5.9.

In a particularly preferred embodiment of the present invention, the reaction temperature is at most 98° C., in particular at most 95° C., in particular at most 91° C., in particular at most 85° C., in particular at most 82° C., in particular at most 79° C., in particular at most 78° C., in particular at most 72° C., in particular at most 70° C., in particular at most 65° C., in particular at most 60° C., in particular at most 55° C.

In a particularly preferred embodiment of the present invention, the reaction temperature in method step b) is from 30 to 100° C., in particular from 30 to 98° C., in particular from 32 to 95° C., in particular from 30 to 91° C., in particular from 30 to 79° C., in particular 30 to 75° C., in particular 30 to 70° C., in particular 30 to 60° C., in particular 30 to 50° C., in particular 40 to 100° C., in particular 40 to 98° C., in particular 40 to 95° C., in particular 40 to 91° C., in particular 40 to 79° C., in particular 40 to 75° C., in particular 40 to 70° C., in particular 40 to 60° C., in particular 45 to 100° C., in particular 45 to 98° C., in particular 45 to 95° C., in particular 45 to 91° C., in particular 45 to 79° C., in particular 45 to 75° C., in particular 45 to 70° C., in particular 45 to 60° C., in particular 50 to 100° C., in particular 50 to 98° C., in particular 50 to 95° C., in particular 50 to 91° C., in particular 50 to 80° C., in particular 50 to 70° C., in particular 50 to 60° C., in particular 55 to 100° C., in particular 55 to 98° C., in particular 55 to 95° C., in particular 55 to 91° C., in particular 55 to 79° C., in particular 55 to 75° C., in particular 55 to 70° C., in particular 60 to 100° C., in particular 60 to 98° C., in particular 60 to 95° C., in particular 60 to 91° C., in particular 60 to 79° C., in particular 60 to 75° C., in particular 60 to 70° C., in particular 65 to 100° C., in particular 65 to 98° C., in particular 65 to 95° C., in particular 65 to 91° C., in particular 65 to 85° C., in particular 65 to 79° C., in particular 68 to 100° C., in particular 68 to 98° C., in particular 68 to 95° C., in particular 68 to 91° C., in particular 68 to 79° C., in particular 70 to 100° C., in particular 70 to 98° C., in particular 70 to 95° C., in particular 70 to 91° C., in particular 70 to 79° C.

In a particularly preferred embodiment of the present invention, the reaction temperature in method step b) is 70 to 95° C.

In a particularly preferred embodiment, the carbohydrate mixture is preheated to 30 to 70° C. in method step a1) and a reaction temperature of 70 to 95° C. is set in method step b).

In a preferred embodiment of the invention, the isomaltulose in method step b) is converted to 1,6-GPS (6-O-α-D-Glucopyranosyl-D-sorbitol) and 1,1-GPM (1-O-α-D-glucopyranosyl-D-mannitol) in method step b) with a conversion rate of 99.5 to 100 mol-%, in particular 99.9 to 100 mol-%.

In a preferred embodiment of the invention, the isomaltulose in method step b) is converted with a selectivity of 98 to 100 mol %, in particular 99 to 100 mol %, in particular 99.5 to 100 mol-%, to 1,6-GPS (6-O-α-D-glucopyranosyl-D-sorbitol) and 1,1-GPM (1-O-α-D-glucopyranosyl-D-mannitol).

In a particularly preferred embodiment of the invention, the hydrogen pressure is 16.5 to 21.0 MPa, the reaction temperature is 30 to 95° C., the space velocity is 0.3 to 0.9 h⁻¹ and the pH is 2.5 to 6.0.

In a particularly preferred embodiment of the invention, the hydrogen pressure is 16.5 to 21.0 MPa, the reaction temperature is 50 to 95° C., the space velocity is 0.3 to 0.9 h⁻¹ and the pH is 2.5 to 6.0.

In a particularly preferred embodiment of the invention, the hydrogen pressure is 16.5 to 21.0 MPa, the reaction temperature is 80 to 95° C., the space velocity is 0.3 to 0.9 h⁻¹ and the pH is 2.5 to 6.0, in particular after preheating in a method step a1) to 30 to 80° C.

In a particularly preferred embodiment of the invention, the hydrogen pressure is 16.5 to 21.0 MPa, the reaction temperature is 70 to 95° C., the space velocity is 0.3 to 0.9 and the pH is 2.5 to 6.0, in particular after preheating in a method step a1) to 30 to 75° C.

In a particularly preferred embodiment of the invention, the hydrogen pressure is 16.5 to 21.0 MPa, the reaction temperature is 70 to 95° C., the space velocity is 0.3 to 0.9 h⁻¹ and the pH is 2.5 to 6.0, in particular after preheating in a method step a1) to 30 to 70° C.

In a particularly preferred embodiment of the invention, the hydrogen pressure is 16.5 to 21.0 MPa, the reaction temperature is 30 to 79° C., the space velocity is 0.3 to 0.9 h⁻¹ and the pH is 2.5 to 6.0.

In a particularly preferred embodiment of the invention, the hydrogen pressure is 16.5 to 21.0 MPa, the reaction temperature is 50 to 79° C., the space velocity is 0.3 to 0.9 h⁻¹ and the pH is 2.5 to 6.0.

In a particularly preferred embodiment of the invention, the hydrogen pressure is 16.5 to 21.0 MPa, the reaction temperature is 70 to 79° C., the space velocity is 0.3 to 0.9 h⁻¹ and the pH is 2.5 to 6.0, in particular after preheating in a method step a1) to 30 to 75° C.

In a particularly preferred embodiment of the invention, the hydrogen pressure is 16.5 to 21.0 MPa, the reaction temperature is 70 to 79° C., the space velocity is 0.3 to 0.9 h⁻¹ and the pH is 2.5 to 6.0, in particular after preheating in a method step a1) to 30 to 70° C.

In a particularly preferred embodiment of the invention, the hydrogen pressure is 16.5 to 18.0 MPa, the reaction temperature is 30 to 95° C., the space velocity is 0.3 to 0.9 h⁻¹ and the pH is 2.5 to 6.0.

In a particularly preferred embodiment of the invention, the hydrogen pressure is 16.5 to 18.0 MPa, the reaction temperature is 50 to 95° C., the space velocity is 0.3 to 0.9 h⁻¹ and the pH is 2.5 to 6.0.

In a particularly preferred embodiment of the invention, the hydrogen pressure is 16.5 to 18.0 MPa, the reaction temperature is 70 to 95° C., the space velocity is 0.3 to 0.9 h⁻¹ and the pH is 2.5 to 6.0, in particular after preheating in a method step a1) to 30 to 80° C.

In a particularly preferred embodiment of the invention, the hydrogen pressure is 16.5 to 18.0 MPa, the reaction temperature is 70 to 95° C., the space velocity is 0.3 to 0.9 h⁻¹ and the pH is 2.5 to 6.0, in particular after preheating in a method step a1) to 30 to 75° C.

In a particularly preferred embodiment of the invention, the hydrogen pressure is 16.5 to 18.0 MPa, the reaction temperature is 70 to 95° C., the space velocity is 0.3 to 0.9 h⁻¹ and the pH is 2.5 to 6.0, in particular after preheating in a method step a1) to 30 to 70° C.

In a particularly preferred embodiment of the invention, the hydrogen pressure is 16.5 to 18.0 MPa, the reaction temperature is 30 to 79° C., the space velocity is 0.3 to 0.9 h⁻¹ and the pH is 2.5 to 6.0.

In a particularly preferred embodiment of the invention, the hydrogen pressure is 16.5 to 18.0 MPa, the reaction temperature is 50 to 79° C., the space velocity is 0.3 to 0.9 h⁻¹ and the pH is 2.5 to 6.0.

In a particularly preferred embodiment of the invention, the hydrogen pressure is 16.5 to 18.0 MPa, the reaction temperature is 70 to 79° C., the space velocity is 0.3 to 0.9 h⁻¹ and the pH is 2.5 to 6.0, in particular after preheating in a method step a1) to 30 to 75° C.

In a particularly preferred embodiment of the invention, the hydrogen pressure is 16.5 to 18.0 MPa, the reaction temperature is 70 to 79° C., the space velocity is 0.3 to 0.9 h⁻¹ and the pH is 2.5 to 6.0, in particular after preheating in a method step a1) to 30 to 70° C.

In a particularly preferred embodiment of the invention, the hydrogen pressure is 16.5 to 21.0 MPa, the reaction temperature is 30 to 95° C., the space velocity is 0.25 to 0.9 h⁻¹ and the pH is 3.4 to 5.9.

In a particularly preferred embodiment of the invention, the hydrogen pressure is 16.5 to 21.0 MPa, the reaction temperature is 50 to 95° C., the space velocity is 0.25 to 0.9 h⁻¹ and the pH is 3.4 to 5.9.

In a particularly preferred embodiment of the invention, the hydrogen pressure is 16.5 to 21.0 MPa, the reaction temperature is 70 to 95° C., the space velocity is 0.25 to 0.9 h⁻¹ and the pH is 3.4 to 5.9, in particular after preheating in a method step a1) to 30 to 80° C.

In a particularly preferred embodiment of the invention, the hydrogen pressure is 16.5 to 21.0 MPa, the reaction temperature is 70 to 95° C., the space velocity is 0.25 to 0.9 h⁻¹ and the pH is 3.4 to 5.9, in particular after preheating in a method step a1) to 30 to 75° C.

In a particularly preferred embodiment of the invention, the hydrogen pressure is 16.5 to 21.0 MPa, the reaction temperature is 70 to 95° C., the space velocity is 0.25 to 0.9 h⁻¹ and the pH is 3.4 to 5.9, in particular after preheating in a method step a1) to 30 to 70° C.

In a particularly preferred embodiment of the invention, the hydrogen pressure is 16.5 to 21.0 MPa, the reaction temperature is 30 to 79° C., the space velocity is 0.25 to 0.9 h⁻¹ and the pH is 3.4 to 5.9.

In a particularly preferred embodiment of the invention, the hydrogen pressure is 16.5 to 21.0 MPa, the reaction temperature is 50 to 79° C., the space velocity is 0.25 to 0.9 h⁻¹ and the pH is 3.4 to 5.9.

In a particularly preferred embodiment of the invention, the hydrogen pressure is 16.5 to 21.0 MPa, the reaction temperature is 70 to 79° C., the space velocity is 0.25 to 0.9 h⁻¹ and the pH is 3.4 to 5.9, in particular after preheating in a method step a1) to 30 to 75° C.

In a particularly preferred embodiment of the invention, the hydrogen pressure is 16.5 to 21.0 MPa, the reaction temperature is 70 to 79° C., the space velocity is 0.25 to 0.9 h⁻¹ and the pH is 3.4 to 5.9, in particular after preheating in a method step a1) to 30 to 70° C.

In a particularly preferred embodiment of the invention, the hydrogen pressure is 16.5 to 18.0 MPa, the reaction temperature is 30 to 95° C., the space velocity is 0.25 to 0.9 h⁻¹ and the pH is 3.4 to 5.9.

In a particularly preferred embodiment of the invention, the hydrogen pressure is 16.5 to 18.0 MPa, the reaction temperature is 50 to 95° C., the space velocity is 0.25 to 0.9 h⁻¹ and the pH is 3.4 to 5.9.

In a particularly preferred embodiment of the invention, the hydrogen pressure is 16.5 to 18.0 MPa, the reaction temperature is 70 to 95° C., the space velocity is 0.25 to 0.9 h⁻¹ and the pH is 3.4 to 5.9, in particular after preheating in a method step a1) to 30 to 80° C.

In a particularly preferred embodiment of the invention, the hydrogen pressure is 16.5 to 18.0 MPa, the reaction temperature is 70 to 95° C., the space velocity is 0.25 to 0.9 h⁻¹ and the pH is 3.4 to 5.9, in particular after preheating in a method step a1) to 30 to 75° C.

In a particularly preferred embodiment of the invention, the hydrogen pressure is 16.5 to 18.0 MPa, the reaction temperature is 70 to 95° C., the space velocity is 0.25 to 0.9 h⁻¹ and the pH is 3.4 to 5.9, in particular after preheating in a method step a1) to 30 to 70° C.

In a particularly preferred embodiment of the invention, the hydrogen pressure is 16.5 to 18.0 MPa, the reaction temperature is 30 to 79° C., the space velocity is 0.25 to 0.9 h⁻¹ and the pH is 3.4 to 5.9.

In a particularly preferred embodiment of the invention, the hydrogen pressure is 16.5 to 18.0 MPa, the reaction temperature is 50 to 79° C., the space velocity is 0.25 to 0.9 h⁻¹ and the pH is 3.4 to 5.9.

In a particularly preferred embodiment of the invention, the hydrogen pressure is 16.5 to 18.0 MPa, the reaction temperature is 70 to 79° C., the space velocity is 0.25 to 0.9 h⁻¹ and the pH is 3.4 to 5.9, in particular after preheating in a method step a1) to 30 to 75° C.

In a particularly preferred embodiment of the invention, the hydrogen pressure is 16.5 to 18.0 MPa, the reaction temperature is 70 to 79° C., the space velocity is 0.25 to 0.9 h⁻¹ and the pH is 3.4 to 5.9, in particular after preheating in a method step a1) to 30 to 70° C.

In a particularly preferred embodiment of the invention, the hydrogen pressure is 16.5 to 21.0 MPa, the reaction temperature is 30 to 95° C., the space velocity is 0.25 to 1.5 h⁻¹ and the pH is 2.5 to 6.0.

In a particularly preferred embodiment of the invention, the hydrogen pressure is 16.5 to 21.0 MPa, the reaction temperature is 50 to 95° C., the space velocity is 0.25 to 1.5 h⁻¹ and the pH is 2.5 to 6.0.

In a particularly preferred embodiment of the invention, the hydrogen pressure is 16.5 to 21.0 MP a, the reaction temperature is 70 to 95° C., the space velocity is 0.25 to 1.5 h⁻¹ and the pH is 2.5 to 6.0, in particular after preheating in a method step a1) to 30 to 80° C.

In a particularly preferred embodiment of the invention, the hydrogen pressure is 16.5 to 21.0 MPa, the reaction temperature is 70 to 95° C., the space velocity is 0.25 to 1.5 h⁻¹ and the pH is 2.5 to 6.0, in particular after preheating in a method step a1) to 30 to 75° C.

In a particularly preferred embodiment of the invention, the hydrogen pressure is 16.5 to 21.0 MPa, the reaction temperature is 70 to 95° C., the space velocity is 0.25 to 1.5 h⁻¹ and the pH is 2.5 to 6.0, in particular after preheating in a method step a1) to 30 to 70° C.

In a particularly preferred embodiment of the invention, the hydrogen pressure is 16.5 to 21.0 MPa, the reaction temperature is 30 to 79° C., the space velocity is 0.25 to 1.5 h⁻¹ and the pH is 2.5 to 6.0.

In a particularly preferred embodiment of the invention, the hydrogen pressure is 16.5 to 21.0 MP a, the reaction temperature is 50 to 79° C., the space velocity is 0.25 to 1.5 h⁻¹ and the pH is 2.5 to 6.0.

In a particularly preferred embodiment of the invention, the hydrogen pressure is 16.5 to 21.0 MPa, the reaction temperature is 70 to 79° C., the space velocity is 0.25 to 1.5 h⁻¹ and the pH is 2.5 to 6.0, in particular after preheating in a method step a1) to 30 to 75° C.

In a particularly preferred embodiment of the invention, the hydrogen pressure is 16.5 to 21.0 MPa, the reaction temperature is 70 to 79° C., the space velocity is 0.25 to 1.5 h⁻¹ and the pH is 2.5 to 6.0, in particular after preheating in a method step a1) to 30 to 70° C.

In a particularly preferred embodiment of the invention, the hydrogen pressure is 16.5 to 18.0 MPa, the reaction temperature is 30 to 95° C., the space velocity is 0.25 to 1.5 h⁻¹ and the pH is 2.5 to 6.0.

In a particularly preferred embodiment of the invention, the hydrogen pressure is 16.5 to 18.0 MPa, the reaction temperature is 50 to 95° C., the space velocity is 0.25 to 1.5 h⁻¹ and the pH is 2.5 to 6.0.

In a particularly preferred embodiment of the invention, the hydrogen pressure is 16.5 to 18.0 MPa, the reaction temperature is 70 to 95° C., the space velocity is 0.25 to 1.5 h⁻¹ and the pH is 2.5 to 6.0, in particular after preheating in a method step a1) to 30 to 80° C.

In a particularly preferred embodiment of the invention, the hydrogen pressure is 16.5 to 18.0 MPa, the reaction temperature is 70 to 95° C., the space velocity is 0.25 to 1.5 h⁻¹ and the pH is 2.5 to 6.0, in particular after preheating in a method step a1) to 30 to 75° C.

In a particularly preferred embodiment of the invention, the hydrogen pressure is 16.5 to 18.0 MPa, the reaction temperature is 70 to 95° C., the space velocity is 0.25 to 1.5 h⁻¹ and the pH is 2.5 to 6.0, in particular after preheating in a method step a1) to 30 to 70° C.

In a particularly preferred embodiment of the invention, the hydrogen pressure is 16.5 to 18.0 MPa, the reaction temperature is 30 to 79° C., the space velocity is 0.25 to 1.5 h⁻¹ and the pH is 2.5 to 6.0.

In a particularly preferred embodiment of the invention, the hydrogen pressure is 16.5 to 18.0 MPa, the reaction temperature is 50 to 79° C., the space velocity is 0.25 to 1.5 h⁻¹ and the pH is 2.5 to 6.0.

In a particularly preferred embodiment of the invention, the hydrogen pressure is 16.5 to 18.0 MPa, the reaction temperature is 70 to 79° C., the space velocity is 0.25 to 1.5 h⁻¹ and the pH is 2.5 to 6.0, in particular after preheating in a method step a1) to 30 to 75° C.

In a particularly preferred embodiment of the invention, the hydrogen pressure is 16.5 to 18.0 MPa, the reaction temperature is 70 to 79° C., the space velocity is 0.25 to 1.5 h⁻¹ and the pH is 2.5 to 6.0, in particular after preheating in a method step a1) to 30 to 70° C.

In connection with the present invention, a “ruthenium-based catalyst” is understood to mean a catalyst which comprises elemental ruthenium and/or ruthenium oxide and/or a ruthenium-containing compound, for example a ruthenium salt.

In particular, the catalyst comprises 0.05 to 20.00% by weight, in particular 0.05 to 5.00% by weight, 0.10 to 20.00% by weight, in particular 0.30 to 10.00% by weight, in particular 0.50 to 5.00% by weight ruthenium (in each case based on elemental ruthenium and the dry weight of the catalyst).

In a preferred embodiment, the ruthenium oxide can be sesquioxide, dioxide or tetraoxide.

In a preferred embodiment, the ruthenium salt can be ruthenium nitrosyl nitrate, ruthenium acetylacetonate, barium perruthenate, sodium perruthenate, a ruthenate such as magnesium, strontium, calcium, silver, barium, potassium or sodium ruthenate, a perruthenate such as sodium or potassium perruthenate, a Ruthenium halide such as ruthenium dichloride, ruthenium trichloride, ruthenium tetrachloride, ruthenium pentafluoride, a ruthenium sulfide such as ruthenium disulfide or a chlorine salt of ruthenium such as potassium chloroperruthenate.

In connection with the present invention, the term “ruthenium,” unless stated otherwise, is understood to mean elemental ruthenium or a ruthenium-containing compound.

The ruthenium-based catalyst can be a monometallic catalyst, in particular contain ruthenium alone as the catalytically active metal, or in a further embodiment also be a bimetallic catalyst which contains another metal in addition to ruthenium, for example nickel, palladium, platinum, iridium, cobalt, rhenium, osmium, gold, silver, or copper.

If, according to the preferred embodiment, a bimetallic catalyst is present, in a preferred embodiment this can contain 5.00 to 95.00% by weight ruthenium, preferably at least 50.00% by weight, in particular at least 55% by weight, ruthenium (in each case based on elemental catalytically active metals and total weight of the catalytically active metals of the catalyst).

In a particularly preferred embodiment of the present invention, the ruthenium-based catalyst is a catalyst immobilized on a carrier.

In a particularly preferred embodiment of the present invention, the carrier is an acidic carrier, in particular an intrinsically acidic carrier, that is to say a carrier which, due to its chemical composition, has an acidic effect or a carrier which has an acidic effect by applying acidic functions.

In a particularly preferred embodiment of the present invention, this carrier is carbon, a metal oxide, in particular aluminum oxide (Al₂O₃), titanium dioxide (TiO₂) or silicon dioxide (SiO₂), zirconium dioxide (ZrO₂), or a zeolite, for example a zeolite of the H—Y type.

In a particularly preferred embodiment, the catalyst used according to the invention can be prepared in a known manner, as described in the textbook “Technical Catalysis—An Introduction” by Jens Hagen (VCH Weinheim, 1996), by first impregnating or coating the catalyst carrier with a solution of a ruthenium salt, then the carrier treated in this way is dried, heated and exposed to a reducing gas stream.

In method step b) according to the invention, the prepared carbohydrate mixture is converted to isomalt and can be obtained, in particular isolated, from the aqueous reaction medium in the subsequent method step c). Conventional isolation methods, for example crystallization method, can be used for this purpose.

In a preferred embodiment, the isomalt can be obtained in method step c) in solid, dry form by appropriate isolation, e.g. crystallization method and drying methods.

In a preferred embodiment, it can be provided that the isomalt present in liquid form is dried by means of evaporators, dryers, in particular spray dryers, falling film evaporators, drum dryers or other conventional devices.

In a further embodiment, in method step c) the isomalt can be obtained in liquid, for example dissolved or suspended, form, in particular by concentration steps, for example evaporation steps or membrane methods.

In a preferred embodiment, the isomalt obtained in method step c) can be in liquid, semi-liquid or dry form, in particular in crystalline form.

The present invention also relates to isomalt, which can be produced by one of the methods according to the invention, in particular obtained in method step c).

In a preferred embodiment, the isomalt produced according to the invention in method step c) is a sugar substitute which comprises 1,6-GPS and 1,1-GPM as main components, in particular at least 86.00% by weight 1,6-GPS and 1,1-GPM (based on total DM of the isomalt). In a preferred embodiment, this isomalt is a mixture containing 1,6-GPS and 1,1-GPM with a 1,6-GPS to 1,1-GPM ratio of >1, in particular 55 to 62% by weight 1,6-GPS and 38 to 45% by weight 1,1-GPM (based on DM of the total amount of 1,6-GPS and 1,1-GPM in isomalt).

In a particularly preferred embodiment, the isomalt produced according to the invention is a JECFA-compliant isomalt which contains at least 98.00% by weight hydrogenated mono- and disaccharides, namely 1,6-GPS, 1,1-GPM, 1,1-GPS, sorbitol and mannitol, and at most 2.00% by weight secondary components, wherein in the isomalt at least 86% by weight 1,6-GPS and 1,1-GPM, 0 to 0.30% by weight reducing sugars and at most 0.50% by weight, in particular 0.01 to 0.50% by weight, sucrose are present (in each case based on the total DM of the isomalt).

Such a JECFA-compliant isomalt is, in a preferred embodiment, a mixture containing 1,6-GPS and 1,1-GPM with a 1,6-GPS to 1,1-GPM ratio of >1, in particular 55 to 62% by weight 1,6-GPS and 38 to 45% by weight 1,1-GPM (based on DM of the total amount of 1,6-GPS and 1,1-GPM in isomalt).

Isomalt, in particular JECFA-compliant isomalt, can be produced in a preferred embodiment from an isomaltulose- and sucrose-containing carbohydrate mixture provided in method step a), which contains 86.00 to 99.99% by weight, in particular 98.00 to 99.99% by weight isomaltulose and at most 0.50% by weight, in particular 0.01 to 0.50% by weight, sucrose (in each case DM (dry matter), based on the total DM of the carbohydrate mixture).

Isomalt, in particular JECFA-compliant isomalt, can be produced in a preferred embodiment from an isomaltulose- and sucrose-containing carbohydrate mixture provided in method step a), which contains 86.00 to 99.99% by weight, in particular 98.00 to 99.99% by weight isomaltulose and at most 0.50% by weight, in particular 0.01 to 0.50% by weight, sucrose and optionally, adding up to 100% by weight of the carbohydrate mixture (DM), trehalulose, isomaltose, glucose, fructose and/or oligomers of carbohydrates, wherein if oligomers of carbohydrates are present, these are present in an amount of at most 0.5% by weight (in each case DM (dry matter), based on total DM of the carbohydrate mixture).

Isomalt, in particular a JECFA-compliant isomalt, can in a preferred embodiment also be produced from an isomaltulose- and sucrose-containing carbohydrate mixture provided in method step a), which contains 75.00 to 99.49% by weight isomaltulose, 0.50 to 24.99% by weight trehalulose and at most 0.50% by weight, in particular 0.01 to 0.50% by weight, sucrose (in each case DM based on total DM of the carbohydrate mixture).

Isomalt, in particular a JECFA-compliant isomalt, can in a preferred embodiment also be produced from an isomaltulose and sucrose-containing carbohydrate mixture provided in method step a) which contains 75.00 to 99.49% by weight isomaltulose, 0.50 to 24.99% by weight trehalulose and at most 0.50% by weight, in particular 0.01 to 0.50% by weight, sucrose and, optionally adding up to 100% by weight of the carbohydrate mixture (DM), isomaltose, glucose, fructose and/or oligomers of carbohydrates, wherein if oligomers of carbohydrates are present, these are present in an amount of at most 0.50% by weight (in each case DM (dry matter), based on the total DM of the carbohydrate mixture).

Isomalt, in particular a JECFA-compliant isomalt, can in a preferred embodiment also be produced from an isomaltulose- and sucrose-containing carbohydrate mixture provided in method step a), which contains 75.00 to 86.00% by weight isomaltulose, 13.99 to 24.99% by weight trehalulose and at most 0.50% by weight, in particular 0.01 to 0.50% by weight, sucrose (in each case DM based on total DM of the carbohydrate mixture).

Isomalt, in particular a JECFA-compliant isomalt, can in a preferred embodiment also be produced from an isomaltulose- and sucrose-containing carbohydrate mixture provided in method step a), which contains 75.00 to 86.00% by weight isomaltulose, 13.99% to 24.99% by weight trehalulose and at most 0.50% by weight, in particular 0.01 to 0.50% by weight, sucrose and, optionally adding up to 100% by weight of the carbohydrate mixture (DM), isomaltose, glucose, fructose and/or oligomers of carbohydrates, wherein if oligomers of carbohydrates are present, these are present in an amount of at most 0.50% by weight (in each case DM (dry matter), based on the total DM of the carbohydrate mixture).

The present invention also relates to the production of a highly pure isomalt, in particular the isomalt obtained in method step c), which preferably contains at least 98.00% by weight hydrogenated mono- and disaccharides, namely 1,6-GPS, 1,1-GPM, 1,1-GPS, sorbitol and mannitol, and at most 2.00% by weight secondary components, wherein at least 98.00% by weight 1,6-GPS and 1,1-GPM, 0 to 0.50% by weight sorbitol, 0 to 0.50% by weight mannitol, at most 0.50% by weight, in particular 0.01 to 0.50% by weight sucrose, and 0 to 0.30% by weight, in particular 0.01 to 0.30% by weight reducing sugars are present in the isomalt, wherein individual secondary components optionally present in this isomalt each are present in an amount of 0 to 0.50% by weight, and wherein the sum of all reducing and non-reducing sugars is at most 0.50% by weight (DM, dry matter in each case based on total DM isomalt).

In a preferred embodiment, a highly pure isomalt can be produced from an isomaltulose- and sucrose-containing carbohydrate mixture provided in method step a), which contains 98.00 to 99.99% by weight isomaltulose and at most 0.50% by weight, in particular 0.01 to 0.50% by weight sucrose (in each case DM (dry matter), based on total DM of the carbohydrate mixture).

Such a highly pure isomalt can be produced in a preferred embodiment from an isomaltulose- and sucrose-containing carbohydrate mixture provided in method step a), which contains 98.00 to 99.99% by weight isomaltulose and at most 0.50% by weight, in particular 0.01 to 0.50% by weight sucrose and optionally, adding up to 100% by weight of the carbohydrate mixture (DM), trehalulose, isomaltose, glucose, fructose and/or oligomers of carbohydrates, wherein if glucose, fructose and/or oligomers of carbohydrates are present, these are each present in an amount of at most 0.5% by weight (in each case DM (dry matter), based on the total DM of the carbohydrate mixture).

In a preferred embodiment, this highly pure isomalt is a mixture containing 1,6-GPS and 1,1-GPM with a 1,6-GPS to 1,1-GPM ratio of >1, in particular 55 to 62% by weight 1,6-GPS and 38 to 45% by weight 1,1-GPM (based on DM of the total amount of 1,6-GPS and 1,1-GPM in isomalt).

In a particularly preferred embodiment, the JEFCA-compliant or highly pure isomalt obtained in method step c) contains at least 98.80% by weight, preferably at least 99.00% by weight, preferably at least 99.10% by weight hydrogenated mono- and disaccharides, in particular disaccharide alcohols (each based on DM of the isomalt).

In a particularly preferred embodiment, the isomalt provided comprises at least 98.00, in particular at least 98.20% by weight, preferably at least 98.50% by weight, preferably at least 98.60% by weight, preferably at least 98.70% by weight 1,6-GPS, 1,1-GPM and 1,1-GPS, preferably 1,6-GPS and 1,1-GPM (each based on the total dry weight of the isomalt).

In a preferred embodiment, the isomalt obtained in method step c) preferably comprises 0.01 to 0.45% by weight, in particular 0.01 to 0.42% by weight sucrose, in particular 0.01 to 0.49% by weight, in particular 0.01 to 0.20% by weight, in particular 0.01 to 0.04% by weight, in particular 0.10 to 0.50% by weight, in particular 0.02 to 0.04% by weight, in particular 0.02 to 0.03% by weight sucrose (each based on the total dry weight of the isomalt).

In a particularly preferred embodiment, the isomalt obtained in method step c) comprises at least 98.00% by weight, preferably at least 98.20% by weight, preferably at least 98.50% by weight, disaccharide alcohols, in particular 1,6-GPS and 1,1-GPM and optionally 1,1-GPS, and 0.01 to 0.40% by weight, preferably 0.01 to 0.03% by weight, sucrose, 0.01 to 0.30% by weight % reducing sugars, at most 0.50% by weight sorbitol and at most 0.50% by weight mannitol, wherein the sum of all sugars, in particular isomaltulose, isomaltose, sucrose, fructose and glucose are at most 0.50% by weight (DM, dry matter based on DM isomalt).

In one embodiment, the isomalt obtained in method step c) preferably has at least 98.00% by weight 1,6-GPS and 1,1-GPM and 0.01 to 0.05% by weight sucrose (DM, each based on DM isomalt).

In a preferred embodiment, isomalt is particularly advantageously provided which is enriched in 1,6-GPS. In relation to 1,1-GPM, 1,6-GPS has a stronger sweetening power and a higher solubility in water. In a particularly preferred embodiment of the present invention, the weight ratio of 1,6-GPS to 1,1-GPM in the isomalt obtained is >1.

In a particularly preferred embodiment, the isomalt provided in method step c), in particular the JECFA-compliant isomalt provided, in particular the highly pure isomalt, is an isomalt with a 1,6-GPS to 1,1-GPM ratio of >1, namely 55 to 62% by weight 1,6-GPS and 38 to 45% by weight 1,1-GPM (based on DM of the total amount of 1,6-GPS and 1,1-GPM in the isomalt).

In a particularly preferred embodiment, a 1,6-GPS-enriched isomalt is provided in method step c), in particular one that contains more than 57.00 to 99.00% by weight, in particular 58.00 to 99.00% by weight, 1,6-GPS and less than 43.00 to 1.00% by weight, in particular 42.00 to 1.00% by weight, 1,1-GPM, in particular 75.00 to 80.00% by weight 1,6-GPS and 25.00 to 20.00% by weight 1,1-GPM (in each case based on dry matter (DM) of the total amount of 1,6-GPS and 1,1-GPM).

In a further preferred embodiment, isomalt is provided in method step c) which contains 43.00 to 57.00% by weight 1,6-GPS and 57.00 to 43.00% by weight 1,1-GPM (each based on dry matter (DM) of the total amount of 1,6-GPS and 1,1-GPM).

In a particularly preferred embodiment, the isomalt provided in method step c) does not comprise any glucose.

In a particularly preferred embodiment, the isomalt provided does not comprise any fructose.

In a preferred embodiment, the isomalt provided does not comprise any sorbitol. In a preferred embodiment, the isomalt according to the invention does not comprise any mannitol.

In a preferred embodiment, the isomalt provided does not comprise any sorbitol, mannitol, glucose or fructose.

In a preferred embodiment of the invention, the isomalt provided in method step c) comprises less than 0.01% by weight, in particular no isomaltulose.

In a particularly preferred embodiment, the isomalt obtained in method step c) comprises at most 0.5% by weight, in particular at most 0.2% by weight, in particular at most 0.15% by weight, in particular at most 0.1% by weight %, in particular at most 0.05% by weight, in particular at most 0.01% by weight glucose (based on the total weight of the dry matter of the isomalt).

In a particularly preferred embodiment, the isomalt provided comprises at most 0.5% by weight, in particular at most 0.2% by weight, in particular at most 0.15% by weight, in particular at most 0.1% by weight, in particular at most 0.05% by weight, in particular at most 0.01% by weight fructose (based on the total weight of the dry matter of the isomalt).

In a preferred embodiment, the isomalt obtained in method step c) comprises at most 0.5% by weight, in particular at most 0.2% by weight, in particular at most 0.15% by weight, in particular at most 0.1% by weight, in particular at most 0.05% by weight, in particular at most 0.01% by weight sorbitol (based on the total weight of the dry matter of the isomalt).

In a preferred embodiment, the isomalt obtained in method step c) comprises at most 0.5% by weight, in particular at most 0.2% by weight, in particular at most 0.15% by weight, in particular at most 0.1% by weight, in particular at most 0.05% by weight, in particular at most 0.01% by weight mannitol (based on the total weight of the dry matter of the isomalt).

In a preferred embodiment, the isomalt obtained in method step c) comprises at most 0.2% by weight, in particular at most 0.1% by weight, in particular at most 0.05% by weight, sorbitol, at most 0.2% by weight, in particular at most 0.1% by weight, in particular at most 0.05% by weight, mannitol, at most 0.2% by weight, in particular at most 0.1% by weight, in particular at most 0.05% by weight, glucose and at most 0.2% by weight, in particular at most 0.1% by weight, in particular at most 0.05% by weight, fructose, wherein the sum of the sugars contained in the isomalt, in particular isomaltulose, isomaltose, sucrose, glucose and fructose, is at most 0.50% by weight (each based on the total weight of the dry matter of the isomalt).

In a particularly preferred embodiment of the present invention, the isomalt obtained in method step c) has 1,6-GPS, 1,1-GPM, and at least one further compound selected from the group consisting of α-D-glucopyranosyl-1,1-D-sorbitol (1,1-GP S), sorbitol and mannitol. In a preferred embodiment, this sorbitol and mannitol contained in the isomalt does not come from a sucrose conversion in method step b), but rather from glucose and fructose optionally contained in the carbohydrate mixture used in method step a).

In a particularly preferred embodiment of the present invention, the isomalt obtained in method step c) contains 50.00 to 60.00% by weight 1,6-GPS, 35.00 to 45.00% by weight 1,1-GPM and optionally 0.10 to 15.00, in particular 0.10 to 1.50% by weight, in particular 0.10 to 1.00% by weight 1,1-GPS, 0.00 to 0.50% by weight, mannitol, preferably no mannitol, 0.00 to 0.50% by weight sorbitol, preferably no sorbitol and 0.01 to 0.50% by weight sucrose, in particular 0.01 to 0.40% by weight, in particular 0.01 to 0.30% by weight, in particular 0.01 to 0.20% by weight, in particular 0.01 to 0.04% by weight, in particular 0.02 to 0.03% by weight, in particular 0.03 to 0.04% by weight, sucrose, and preferably consists of it.

In a particularly preferred embodiment of the present invention, the concentration of sucrose in the isomaltulose- and sucrose-containing carbohydrate mixture provided in step a) is kept constant up to the isomalt obtained in step c) solely by setting the method parameters defined in step b). In a particularly preferred embodiment of the present invention, a method for the production of isomalt is provided, in the context of which the concentration of sucrose in the isomaltulose- and sucrose-containing carbohydrate mixture provided in method step a) is kept constant in method step b), in particular over the entire method, in particular the sucrose content in the isomalt obtained is just as high as the sucrose content in the isomaltulose and sucrose-containing carbohydrate mixture provided.

In a particularly preferred embodiment of the present invention, the concentration of sucrose between the carbohydrate mixture provided in method step a) and the isomalt obtained in method step c) is kept constant, that is, sucrose is not split in method step b) and in particular not split and hydrogenated.

In a particularly preferred embodiment of the present invention, the concentration of sucrose between the carbohydrate mixture provided in step a) and the isomalt obtained in step c) is kept constant solely by adjusting the method parameters, i.e. hydrogen pressure, reaction temperature, space velocity and pH, that is, sucrose is not split in method step b), in particular not split and not hydrogenated.

In connection with the present invention, “keep constant” is understood to mean that the carbohydrate mixture, provided in method step a), has the same sucrose content before the reaction in method step b) as the isomalt obtained after the reaction in method step c). In method step b) the sucrose is therefore not split, in particular not split and not hydrogenated. In a particularly preferred embodiment, the sucrose content is regarded as constant if, using a GC analysis method (GC-FID) with a limit of quantification of 0.01 g sucrose/100 g dry matter, there is no change in the sucrose content during the reaction according to method step b). According to the invention, to determine the sucrose content before and after the reaction according to method step b), the sucrose content (the two values obtained form a measurement pair) is determined several times, in particular 4 times, and an average value for the sucrose content before and an average value for the sucrose content after implementation of method step b) is determined (mean value determination using the upper bound method).

In a preferred embodiment, the sucrose content is considered to be kept constant if the mean value of the sucrose content in the carbohydrate mixture corresponds to the sucrose content in the isomalt obtained, or if no significant difference between the mean values of the sucrose content in the carbohydrate mixture using the difference t-test is found in the isomalt obtained.

The following formula is used for the calculation by means of the difference t-test using the mean values and measured values obtained:

$$τ = \vert \frac{{\overline{x}}_{A} - {\overline{x}}_{B}}{\sqrt{\frac{1}{n - 1} \cdot \sum {( Δ {x}_{i} - Δ \overline{x} )}^{2}}} \vert \cdot \sqrt{n}$$

wherein (x̅_{A}) is the mean value of the component content in the carbohydrate mixture, (x̅_{B}) is the mean value of the component content in the isomalt, (Δx̅) is the difference between the mean values (x̅_{A}-x̅_{B}), (Δxᵢ) is the difference between the respective measurement pairs A (carbohydrate mixture)-B (isomalt), (n) is the number of measurement pairs of the carbohydrate mixture and the isomalt and (τ) is the test variable.

A significant difference (99.9% level of significance) between the mean values of the sucrose content in the carbohydrate mixture and in the isomalt is at n=4 if the test variable (τ) is greater than (t)=12.924 (99.9% level of significance, 3 degrees of freedom).

An optional separation of sucrose, in particular in a method step a0), before the implementation of method step b) remains unaffected.

In a particularly preferred embodiment of the present invention, the isomalt can be used to produce products for human and/or animal consumption or pharmaceutical products.

The present invention therefore also relates to products for human and/or animal consumption or pharmaceutical products containing isomalt according to the invention.

In a preferred embodiment, the product for human consumption is a food or luxury item such as, for example, a confectionery, a filling for confectionery, hard and/or soft candies, a fondant, a yogurt, a pastry, a chewing gum, an ice cream, a dairy product, a fruit preparation, a jam, a jelly or a smoothie.

Hard candies made from at least 96.00% by weight isomalt according to the invention (based on the total weight of the hard candy) have a water absorption of a maximum of 1.30% by weight, in particular 1.20% by weight, in particular 1.10% by weight, in particular 1.00% by weight, in particular 0.90% by weight, in particular 0.80% by weight, when stored open for three days at 30° C. and 65% relative humidity (in each case based on the total weight of the hard candy).

Hard candies made from at least 96.00% by weight of isomalt according to the invention (based on the total weight of the hard candy) have a maximum water absorption of a maximum of 6.0% by weight, in particular 5.50% by weight, in particular 5.00% by weight, in particular 4.50% by weight, in particular 4.00% by weight, in particular 3.50% by weight, in particular 3.00% by weight when stored open for three days at 25° C. and 80% relative humidity (each based on the total weight of the hard candy).

The present invention therefore also relates to hard candies containing isomalt according to the invention, in particular containing at least 96.00% by weight of isomalt according to the invention (based on the total weight of the hard candies), in particular hard candies, which are characterized by the maximum water absorption specified above under the specified conditions.

Further advantageous embodiments result from the dependent claims.

The invention is explained in more detail using the following example.

### EXAMPLES

Hydrogenation of Isomaltulose, Trehalulose and Sucrose-Containing Solutions

The hydrogenations were carried out in a continuous high-pressure reactor using the trickle-bed method. The respective carbohydrate mixtures identified in Table 1 below were dissolved in water and preheated. 1.5% Ru/Al₂O₃ spheres were used as the catalyst. The reaction conditions used in the reactor, pressure, pH, LHSV and reaction temperature, as well as the dry matter content of isomaltulose, trehalulose and sucrose, as well as glucose and fructose in the total DM (dry matter) of the carbohydrate mixture to be hydrogenated (determined by GC-FID) are in each case given in Table 1.

The carbohydrate mixtures used according to reaction numbers 1, 2, 3, 4, 5, 6, 7, 8, 9, 10, 11, 12, 13, 14, 15, 16, 17, 18, 19, 20, 21, 22, and 23 were preheated to temperatures of 47.4° C., 48.2° C., 46.2° C., 55.5° C., 58.5° C., 67.3° C., 45.0° C., 54.4° C., 58.0° C., 63.5° C., 66.5° C., 68.5° C., 70.9° C., 103° C., 103° C., 103° C., 103° C., 68° C., 103° C., 67.3° C., 105° C., 67° C. and 105° C. and then brought to reaction in the reactor at the reaction temperature indicated in Table 1. Reaction numbers 14 to 23 represent non-inventive comparison runs.

TABLE 1

| | | | | | DM | DM | DM | DM | DM | |
|---|---|---|---|---|---|---|---|---|---|---|
| | Hydrogen | pH | | Reaction | isomaltulose | trehalulose | sucrose | fructose | glucose | |
| Reaction | pressure | value | LHSV | temperature | [% by | [% by | [% by | [% by | [% by | DM ref. |
| no. | [MPa] | [−] | [h⁻¹] | [° C.] | weight] | weight] | weight] | weight] | weight] | [g/100 g] |
| 1 | 16.5 | 3.4 | 0.58 | 71.1 | 99.08 | 0.66 | 0.02 | 0.08 | 0.05 | 41.62 |
| 2 | 16.5 | 4.3 | 0.58 | 70.5 | 98.96 | 0.67 | 0.02 | 0.07 | 0.05 | 40.83 |
| 3 | 16.5 | 5.1 | 0.48 | 69.5 | 98.76 | 0.69 | 0.02 | 0.09 | 0.10 | 40.58 |
| 4 | 16.5 | 4.8 | 0.67 | 77.8 | 98.88 | 0.75 | 0.02 | 0.08 | 0.06 | 41.36 |
| 5 | 16.5 | 4.9 | 0.77 | 81.2 | 98.88 | 0.77 | 0.02 | 0.07 | 0.05 | 41.47 |
| 6 | 16.5 | 5.3 | 0.87 | 91.0 | 98.78 | 0.82 | 0.02 | 0.07 | 0.06 | 41.90 |
| 7 | 17.5 | 3.0 | 0.28 | 69.7 | 99.11 | 0.60 | 0.03 | 0.06 | 0.06 | 41.27 |
| 8 | 17.5 | 4.4 | 0.65 | 80.8 | 99.08 | 0.68 | 0.02 | 0.05 | 0.05 | 41.45 |
| 9 | 17.5 | 5.6 | 0.37 | 83.5 | 98.60 | 0.81 | 0.05 | 0.10 | 0.09 | 40.61 |
| 10 | 17.5 | 5.5 | 0.65 | 90.0 | 98.79 | 0.62 | 0.02 | 0.04 | 0.07 | 41.65 |
| 11 | 17.5 | 5.1 | 0.56 | 92.3 | 98.88 | 0.67 | 0.02 | 0.08 | 0.10 | 41.14 |
| 12 | 17.5 | 5.9 | 0.46 | 95.0 | 98.88 | 0.77 | 0.02 | 0.07 | 0.05 | 41.47 |
| 13 | 17.5 | 5.4 | 0.56 | 98.3 | 98.76 | 0.76 | 0.03 | 0.09 | 0.11 | 41.64 |
| 14 | 17.5 | 5.8 | 0.35 | 110.0 | 98.60 | 0.65 | 0.50 | 0.06 | 0.05 | 41.56 |
| 15 | 17.5 | 5.6 | 0.35 | 110.0 | 99.02 | 0.55 | 0.06 | 0.09 | 0.03 | 41.37 |
| 16 | 17.5 | 5.5 | 0.38 | 120.0 | 98.49 | 0.65 | 0.50 | 0.05 | 0.06 | 41.59 |
| 17 | 17.5 | 3.0 | 0.47 | 120.0 | 91.00 | 0.48 | 6.96 | 0.09 | 0.13 | 42.72 |
| 18 | 5.0 | 3.8 | 0.47 | 91.0 | 92.12 | 0.48 | 7.04 | 0.09 | 0.12 | 42.79 |
| 19 | 5.0 | 4.8 | 0.47 | 120.0 | 99.07 | 0.49 | 0.05 | 0.09 | 0.13 | 42.74 |
| 20 | 5.0 | 4.0 | 0.47 | 89.9 | 98.96 | 0.61 | 0.04 | 0.06 | 0.04 | 42.84 |
| 21 | 6.0 | 2.4 | 0.47 | 121 | 98.96 | 0.59 | 0.03 | 0.07 | 0.05 | 41.36 |
| 22 | 6.0 | 2.6 | 0.47 | 90 | 92.27 | 0.54 | 6.85 | 0.10 | 0.07 | 43.08 |
| 23 | 6.0 | 2.5 | 0.47 | 120 | 92.27 | 0.54 | 6.85 | 0.10 | 0.07 | 43.08 |

(% by weight - information based on total dry matter)
(DM ref. is the dry matter content determined by means of a refractometer)
(Reaction numbers 14 to 23 represent non-inventive comparison runs)

TABLE 2a

| | DM | DM | DM | DM | DM | DM | DM | DM |
|---|---|---|---|---|---|---|---|---|
| | 1,6-GPS | 1,1-GPM | 1,1-GPS | mannitol | sorbitol | isomaltulose | trehalulose | sucrose |
| Reaction | [% by | [% by | [% by | [% by | [% by | [% by | [% by | [% by |
| no. | weight] | weight] | weight] | weight] | weight] | weight] | weight] | weight] |
| 1 | 58.96 | 39.91 | 0.33 | 0.03 | 0.09 | <0.01 | <0.01 | 0.02 |
| 2 | 58.72 | 39.98 | 0.27 | 0.02 | 0.07 | <0.01 | <0.01 | 0.02 |
| 3 | 59.45 | 39.40 | 0.31 | 0.03 | 0.09 | <0.01 | <0.01 | 0.02 |
| 4 | 58.65 | 40.06 | 0.34 | 0.03 | 0.08 | <0.01 | <0.01 | 0.02 |
| 5 | 58.63 | 40.25 | 0.26 | 0.02 | 0.05 | <0.01 | <0.01 | 0.02 |
| 6 | 57.98 | 40.67 | 0.37 | 0.03 | 0.08 | <0.01 | <0.01 | 0.02 |
| 7 | 58.01 | 41.13 | 0.30 | 0.03 | 0.02 | <0.01 | <0.01 | 0.03 |
| 8 | 57.60 | 41.47 | 0.34 | 0.02 | 0.07 | 0.03 | <0.01 | 0.02 |
| 9 | 57.52 | 41.46 | 0.30 | 0.04 | 0.10 | 0.03 | <0.01 | 0.05 |
| 10 | 56.88 | 42.00 | 0.35 | 0.08 | 0.04 | 0.04 | <0.01 | 0.02 |
| 11 | 56.47 | 42.23 | 0.38 | 0.03 | 0.09 | <0.01 | <0.01 | 0.02 |
| 12 | 56.71 | 41.75 | 0.34 | 0.02 | 0.05 | <0.01 | <0.01 | 0.02 |
| 13 | 56.11 | 42.15 | 0.41 | 0.04 | 0.10 | <0.01 | <0.01 | 0.03 |
| 14 | 49.24 | 43.55 | 1.66 | 0.20 | 0.28 | <0.01 | <0.01 | 0.22 |
| 15 | 49.11 | 43.95 | 1.73 | 0.05 | 0.12 | <0.01 | <0.01 | 0.02 |
| 16 | 48.16 | 43.10 | 1.90 | 0.19 | 0.30 | <0.01 | <0.01 | 0.18 |
| 17 | 42.29 | 40.22 | 1.78 | 1.87 | 4.27 | <0.01 | <0.01 | 0.24 |
| 18 | 52.20 | 39.78 | 0.18 | 1.07 | 3.31 | 0.05 | <0.01 | 2.63 |
| 19 | 48.05 | 39.73 | 1.78 | 0.04 | 0.16 | <0.01 | <0.01 | 0.02 |
| 20 | 55.1 | 41.78 | 0.36 | 0.03 | 0.10 | 0.01 | <0.01 | 0.01 |
| 21 | 43.82 | 40.20 | 2.36 | 0.49 | 1.01 | <0.01 | <0.01 | <0.01 |
| 22 | 51.63 | 39.44 | 0.43 | 0.41 | 1.48 | <0.01 | <0.01 | 4.66 |
| 23 | 42.49 | 39.40 | 1.95 | 1.79 | 4.30 | <0.01 | <0.01 | 0.02 |

(% by weight - information based on total dry matter)
(Reaction numbers 14 to 23 represent non-inventive comparison runs)

TABLE 2b

| | | | | Sum DM | Total DM of | | | Selectivity |
|---|---|---|---|---|---|---|---|---|
| | | | | 1,1-GPM | hydrogenated | Totalof | | isomalt |
| | DM | DM | | and | mono-and | secondary | | [1,1-GPM |
| | glucose | fructose | | 1,6-GPS | disaccharides | components | Isomaltulose | and 1,6- |
| Reaction | [% by | [% by | DM ref. | [% by | [% by | [% by | conversion | GPS) |
| no. | weight] | weight] | [g/100 g] | weight] | weight] | weight] | [mol %] | [mol %] |
| 1 | <0.01 | <0.01 | 41.90 | 98.87 | 99.32 | 0.68 | >99.99 | 99.20 |
| 2 | <0.01 | <0.01 | 40.98 | 98.70 | 99.06 | 0.94 | >99.99 | 99.15 |
| 3 | <0.01 | <0.01 | 41.13 | 98.85 | 99.29 | 0.71 | >99.99 | 99.51 |
| 4 | <0.01 | <0.01 | 41.66 | 98.71 | 99.15 | 0.85 | >99.99 | 99.24 |
| 5 | <0.01 | <0.01 | 41.21 | 98.88 | 99.21 | 0.79 | >99.99 | 99.41 |
| 6 | <0.01 | <0.01 | 41.79 | 98.65 | 99.12 | 0.88 | >99.99 | 99.28 |
| 7 | <0.01 | <0.01 | 41.70 | 99.14 | 99.55 | 0.45 | >99.99 | 99.44 |
| 8 | <0.01 | <0.01 | 41.34 | 99.07 | 99.50 | 0.50 | 99.97 | 99.40 |
| 9 | <0.01 | <0.01 | 40.96 | 98.98 | 99.42 | 0.58 | >99.99 | 99.80 |
| 10 | <0.01 | <0.01 | 41.77 | 98.88 | 99.34 | 0.66 | 99.96 | 99.51 |
| 11 | <0.01 | <0.01 | 41.68 | 98.70 | 99.20 | 0.80 | >99.99 | 99.23 |
| 12 | <0.01 | <0.01 | 41.06 | 98.46 | 98.88 | 1.12 | >99.99 | 98.99 |
| 13 | <0.01 | <0.01 | 41.96 | 98.26 | 98.81 | 1.19 | >99.99 | 98.91 |
| 14 | <0.01 | 0.01 | 42.34 | 92.79 | 94.93 | 5.07 | >99.99 | 93.56 |
| 15 | <0.01 | <0.01 | 42.27 | 93.06 | 94.96 | 5.04 | >99.99 | 93.43 |
| 16 | <0.01 | <0.01 | 42.52 | 91.26 | 93.65 | 6.35 | >99.99 | 92.12 |
| 17 | 0.11 | 0.03 | 43.04 | 82.51 | 89.75 | 10.25 | >99.99 | 90.14 |
| 18 | 0.04 | 0.20 | 44.49 | 91.98 | 96.54 | 3.46 | 99.95 | 99.26 |
| 19 | <0.01 | <0.01 | 43.04 | 87.78 | 89.76 | 10.24 | >99.99 | 88.09 |
| 20 | <0.01 | <0.01 | 43.18 | 96.88 | 97.35 | 2.65 | 99.99 | 97.32 |
| 21 | 0.10 | 0.03 | 41.96 | 84.01 | 87.87 | 12.13 | >99.99 | 84.40 |
| 22 | 0.19 | 0.23 | 43.73 | 91.07 | 93.39 | 6.61 | >99.99 | 98.12 |
| 23 | 0.04 | 0.04 | 43.64 | 81.89 | 89.94 | 10.06 | <99.99 | 88.23 |

(% by weight data based on total dry matter,
Reaction numbers 14 to 23 represent non-inventive comparison runs.
The sum of hydrogenated mono-and disaccharides was calculated for all reaction runs according to the Joint FAO/WHO Expert Committee on Food Additives (JECFA), Specification for Isomalt (69th JECFA (2008), published in FAO JECFA Monographs 5 (2008)). The sum includes the added weight proportions of 1,1-GPM, 1,6-GPS, 1,1-GPS, mannitoland sorbitol.
The sum of the secondary components is the difference between 100 − the sum of the hydrogenated mono-and disaccharides.
DM ref. is the dry matter content determined by means of a refractometer.)
Tables 2a and 2b show the compositions of the isomalts obtained.

It can be seen that in comparison to the products of reactions 14 to 23 not produced according to the invention, a particularly pure isomalt was produced in reactions 1 to 13. It also shows that surprisingly the sucrose content, starting from the sucrose content of the carbohydrate mixtures used in reaction numbers 1 to 13, remained constant up to the isomalt obtained, whereas the content of 1,6-GPS, 1,1-GPM and 1,1-GPS increased and the isomaltulose content decreased.

1. A method for the continuous production of isomalt from an isomaltulose- and sucrose-containing carbohydrate mixture, comprising the method steps:
a) providing an isomaltulose- and sucrose-containing carbohydrate mixture in an aqueous medium, containing 75.00 to 99.99% by weight isomaltulose and 0.01 to 0.50% by weight sucrose (in each case DM (dry matter), based on the total DM of the carbohydrate mixture), hydrogen and ruthenium-based catalyst;
b) converting the carbohydrate mixture to isomalt by continuously bringing the carbohydrate mixture present in the aqueous medium into contact with the ruthenium-based catalyst and hydrogen at a space velocity of 0.25 to 1.5 h⁻¹, at a hydrogen pressure of 16.0 to 22.0 MPa and a pH of 2.0 to 6.0 to obtain an isomalt-containing product stream while setting a reaction temperature of at most 100° C.; and
c) preserving the isomalt.

2. The method according to claim 1, wherein the isomaltulose and sucrose-containing carbohydrate mixture provided in method step a) was obtained by enzymatic reaction of sucrose or a sucrose-containing starting mixture with a sucrose-glucosylmutase.

3. The method according to claim 2, wherein the isomaltulose and sucrose-containing carbohydrate mixture obtained by enzymatic reaction with a sucrose-glucosyl mutase from sucrose or a sucrose-containing starting mixture is subjected to a method step a0) for reducing the sucrose content to a content of 0.01 to 0.50% by weight sucrose (dry matter based on total dry matter of the carbohydrate mixture).

4. The method according to claim 1, wherein in method step b) a reaction temperature of 70° C. to 95° C., in particular 70° C. to 91° C., is set.

5. The method according to claim 1, wherein after method step a) and before method step b) the carbohydrate mixture provided in method step a) is preheated in a method step a1), in particular to a temperature of 30 to 80° C., in particular 30 to 75° C., in particular 30 to 70° C.

6. The method according to claim 1, wherein the isomaltulose is converted in method step b) at a conversion rate of 99 to 100 mol %, in particular 99.5 to 100 mol %.

7. The method according to claim 1, wherein the isomaltulose in method step b) is converted with a selectivity of 97 to 100 mol %, in particular 98 to 100 mol %, to 1,6-GPS (6-O-α-D-glucopyranosyl-D-sorbitol) and 1,1-GPM (1-O-α-D-glucopyranosyl-D-mannitol).

8. The method according to claim 1, wherein the isomaltulose- and sucrose-containing carbohydrate mixture provided in method step a) comprises 86 to 99.99% by weight isomaltulose, in particular 98 to 99.99% by weight isomaltulose, and 0.01 to 0.50% by weight sucrose (each DM based on total DM of the carbohydrate mixture).

9. The method according to claim 1, wherein the isomaltulose- and sucrose-containing carbohydrate mixture provided in method step a) comprises 75.00 to 99.49% by weight isomaltulose, 0.30 to 24.99% by weight trehalulose and 0.01 to 0.50% by weight sucrose (in each case DM based on total DM of the carbohydrate mixture).

10. The method according to claim 1, wherein the carbohydrate mixture provided in step a) comprises 75 to 86.00% by weight isomaltulose, 0.01 to 0.50% by weight sucrose and 13.99 to 24.99% by weight trehalulose (in each case based on the dry matter of the carbohydrate mixture).

11. The method according to claim 1, wherein the dry matter content of the carbohydrate mixture present in the aqueous medium is 35 to 45% by weight (based on the total weight of the medium).

12. The method according to claim 1, wherein the ruthenium-based catalyst is a catalyst immobilized on a carrier.

13. The method according to claim 1, wherein the ruthenium-based catalyst is a monometallic or a bimetallic catalyst.

14. The method according to claim 12, wherein the carrier is carbon, a metal oxide, in particular Al₂O₃, TiO₂, SiO₂, ZrO₂ or a zeolite.

15. The method according to claim 12, wherein the carrier is Al₂O₃.

16. The method according to claim 1, wherein the ruthenium content of the catalyst is 0.05 to 5.00% by weight.

17. The method according to claim 1, wherein the pH value in method step b) is 2.0 to 5.5.

18. The method according to claim 1, wherein the concentration of sucrose in the carbohydrate mixture provided in step a) is kept constant up to the isomalt obtained in step c) solely by setting the method parameters defined in step b).

19. The method according to claim 1, wherein the isomalt obtained in step c) is at least 98.00% by weight hydrogenated mono- and disaccharides, at least 86.00% by weight 1,6-GPS and 1,1-GPM, 0.01 to 0.50% by weight sucrose and at most 0.30% by weight reducing sugars (in each case DM (dry matter), in each case based on DM isomalt).

20. The method according to claim 1, wherein the isomalt obtained in step c) is at least 98.00% by weight hydrogenated mono- and disaccharides, at least 98.00% by weight 1,6-GPS and 1,1-GPM, 0.01 to 0.50% by weight sucrose, at most 0.30% by weight reducing sugars, 0 to 0.50% by weight sorbitol and 0 to 0.50% by weight mannitol, wherein some of any secondary components present in this isomalt are in each case present in an amount of 0 to 0.50% by weight and wherein the sum of all reducing and non-reducing sugars is at most 0.50% by weight (in each case DM (dry matter), each based on DM isomalt).

21. (canceled)
